(19) United States
(12) Patent Application Publication
(10) Pub. No.: US 2022/0380749 A1
(43) Pub. Date: Dec. 1, 2022

(54) BASE EDITING SYSTEMS FOR ACHIEVING C TO A AND C TO G BASE MUTATION AND APPLICATION THEREOF

(71) Applicant: TIANJIN INSTITUTE OF INDUSTRIAL BIOTECHNOLOGY, CHINESE ACADEMY OF SCIENCES, Tianjin (CN)

(72) Inventors: Xueli ZHANG, Tianjin (CN); Changhao BI, Tianjin (CN); Dongdong ZHAO, Tianjin (CN); Siwei LI, Tianjin (CN)

(21) Appl. No.: 17/636,607

(22) PCT Filed: Aug. 19, 2020
(86) PCT No.: PCT/CN2020/109949

§ 371 Date: Feb. 18, 2022

(30) Foreign Application Priority Data
Aug. 20, 2019 (CN) 2019 10767298.1

(51) Int. Cl. C12N 15/10; C12N 15/90; C12N 9/22; C12N 9/78; C12N 9/24

(52) U.S. Cl. CPC C12N 15/102; C12N 15/902; C12N 15/907; C12N 9/22; C12N 9/78; C12N 9/2497; C12N 2800/80; C12N 2310/20; C12Y 305/04001; C12Y 302/02027

(57) ABSTRACT

The present invention discloses base editing systems for mutating a base C to A and a base C to G and applications thereof. The base editing system for mutating C to A disclosed in the present invention includes cytosine deaminase AID and nCas9 nuclease or includes cytosine deaminase AID, nCas9 nuclease and uracil DNA glycosidase; the base editing system for mutating C to G of the present invention includes cytosine deaminase APOBEC, nCas9 nuclease and uracil DNA glycosidase. The experiments show that a combination of the three base editing systems for mutating C to A, C to T and A to G can realize a mutation of A, T, C or G to any base in both prokaryotes and eukaryotes.

### RELATED APPLICATIONS

The present application is a U.S. National Phase of International Application Number PCT/CN2020/109949 filed Aug. 19, 2020, and claims priority to Chinese Application Number 201910767298.1 filed Aug. 20, 2019.

### INCORPORATION BY REFERENCE

The sequence listing provided in the file entitled SQL_Mod_20220218.txt, which is an ASCII text file that was created on Feb. 18, 2022, and which comprises 112,964 bytes, is hereby incorporated by reference in its entirety.

### TECHNICAL FIELD

The present invention belongs to the field of biotechnology, and particularly relates to base editing systems for mutating a base C to A and a base C to G and applications thereof.

### BACKGROUND

Genome editing refers to the effective design and efficient transformation of cells at a genome scale, and early genome editing mainly used a targeting technology mediated by homologous recombination; however, due to its low efficiency (10⁻⁶-10⁻⁹), a series of engineered endonuclease mediated genome editing technologies had been developed to solve this problem. At present, there are mainly three editing technologies, i.e., artificial nuclease-mediated zinc-finger nucleases (ZFN) technology, transcription activator-like effector nucleases (TALEN) technology and RNA-guided CRISPR/Cas9 nuclease (CRISPR/Cas RGNs) technology, wherein the CRISPR-Cas9 technology is simpler in design, more convenient in operation, and more efficient in gene editing, and it has been successfully applied in the research on genome editing of various target cells.

Although the CRISPR/Cas9 method can precisely edit DNA, it is limited by the low efficiency of homologous DNA repair, thus it is now mainly used for gene knockout and cannot efficiently generate single nucleotide mutations. In order to improve the efficiency of site-directed mutations, the CRISPR system is combined with cytosine deaminase or adenine deaminase to construct a single base editing system, which can realize an accurate replacement from cytosine C to thymine T and from adenine A to guanine G for specific target sites without generating double-stranded DNA breaks. The single base editing system as a new-generation gene editing tool still has certain defects: limited by enzyme functions, currently the single base editing system can only realize the editing of a single base C to T or A to G, which limits the application of the base editing system; therefore, there is an urgent need for constructing a new base editing method and even a system of single base editing from any base to any base.

### SUMMARY

The present invention is intended to realize a mutation of a target base C to A in a genome sequence, improve a base editing efficiency of mutating a target base C to A and mutating the target base C to G, and then realize a mutation from any base to any base.

In order to realize the above-mentioned objectives, the present invention firstly provides a method for mutating a target base C to A in a genome sequence.

The method for mutating a target base C to A in a genome sequence provided by the present invention can be D1) or D2) or D3) or D4) as follows:

D1) the method includes the following steps: using a CRISPR/Cas9 system, cytosine deaminase and uracil DNA glycosidase for single-base editing to mutate a target base C to A;

D2) the method includes the following steps: using a CRISPR/Cas9 system and cytosine deaminase for single-base editing to mutate a target base C to A;

D3) the method includes the following steps: using a CRISPR/Cas9 system, cytosine deaminase AID and uracil DNA glycosidase for single-base editing to mutate a target base C to A;

D4) the method includes the following steps: using a CRISPR/Cas9 system and cytosine deaminase AID for single-base editing to mutate a target base C to A.

Further, the method for mutating a target base C to A in a genome sequence can be d1) or d2) or d3) or d4) as follows:

d1) the method includes the following steps: introducing a coding gene of cytosine deaminase, a coding gene of CRISPR nuclease, a coding gene of uracil DNA glycosidase and a coding sequence of sgRNA into a receptor organism or the cells of a receptor organism, so that the coding gene of cytosine deaminase, the coding gene of CRISPR nuclease, the coding gene of uracil DNA glycosidase and the coding sequence of sgRNA are all expressed to mutate a target base C to A;

d2) the method includes the following steps: introducing a coding gene of cytosine deaminase, a coding gene of CRISPR nuclease and a coding sequence of sgRNA into a receptor organism or the cells of a receptor organism, so that the coding gene of cytosine deaminase, the coding gene of CRISPR nuclease and the coding sequence of sgRNA are all expressed to mutate a target base C to A;

d3) the method includes the following steps: introducing a coding gene of cytosine deaminase AID, a coding gene of nCas9 nuclease, a coding gene of uracil DNA glycosidase and a coding sequence of sgRNA into a receptor organism or the cells of a receptor organism, so that the coding gene of cytosine deaminase AID, the coding gene of nCas9 nuclease, the coding gene of uracil DNA glycosidase and the coding sequence of sgRNA are all expressed to mutate a target base C to A;

d4) the method includes the following steps: introducing a coding gene of cytosine deaminase AID, a coding gene of nCas9 nuclease and a coding sequence of sgRNA into a receptor organism or the cells of a receptor organism, so that the coding gene of cytosine deaminase AID, the coding gene of nCas9 nuclease and the coding sequence of sgRNA are all expressed to mutate a target base C to A;

The sgRNA targets a targeting sequence; the target base C locates in the target sequence.

In d1) and d3), the cytosine deaminase or the cytosine deaminase AID, the CRISPR nuclease or the nCas9 nuclease can be subject to fusion expression or free expression with the uracil DNA glycosidase in the receptor organism or the cells of the receptor organism.

In d2) and d4), the cytosine deaminase or the cytosine deaminase AID, the CRISPR nuclease or the nCas9 nuclease can be subject to fusion expression or free expression in the receptor organism or the cells of the receptor organism.

The cytosine deaminase can be cytosine deaminase from different sources, such as mouse-derived cytosine deaminase APOBEC1 (GenBank: AAH03792.1), human-derived cytosine deaminase APOBEC3A (GenBank: AKE33285.1), lamprey-derived cytosine deaminase pmCDA (Accession: ABO15149.1), etc. Specifically, the cytosine deaminase or the cytosine deaminase AID is lamprey-derived cytosine deaminase pmCDA, and its amino acid sequence is well known in the art; in an example of the present invention, an amino acid sequence of the cytosine deaminase used is as shown in Accession: ABO15149.1 of NCBI.

The uracil DNA glycosidase can be uracil DNA glycosidase from different sources, such as human-derived uracil DNA glycosidase UNG (GenBank: CAG46474.1), yeast-derived uracil DNA glycosidase ung1 (Accession: CAA86634.1), Escherichia coli-derived uracil DNA glycosidase ung (Accession: EGT65982.1), etc. Specifically, the uracil DNA glycosidase is Escherichia coli-derived uracil DNA glycosidase ung, and its amino acid sequence is well known in the art; in an example of the present invention, an amino acid sequence of the Escherichia coli-derived uracil DNA glycosidase used is as shown in Accession: EGT65982.1 of NCBI.

The CRISPR nuclease can be CRISPR nuclease from different sources or mutants thereof, such as Streptococcus pyogenes derived Cas9 nuclease (Accession: Q99ZW2.1) or a mutant thereof, Staphylococcus aureus derived Cas9 nuclease (Accession: AYD60528.1) or a mutant thereof, Francisella tularensis derived cpf1 nuclease (Accession: A0Q7Q2.1) or a mutant thereof. Specifically, the CRISPR nuclease or the nCas9 nuclease is a mutant nCas9-D10A of Cas9, and its amino acid sequence is well known in the art. In an example of the present invention, an amino acid sequence of the CRISPR nuclease used is an amino acid sequence obtained by mutating aspartic acid (D) to alanine (A) in the 10^{th} position from the N-terminal of an amino acid sequence as shown in Accession: Q99ZW2.1 of NCBI.

Even further, in the d3), the coding gene of the cytosine deaminase AID, the coding gene of the nCas9 nuclease and the coding gene of the uracil DNA glycosidase are introduced into the receptor organism or the cells of the receptor organism through a recombinant plasmid A; the recombinant plasmid A expresses a fusion protein composed of cytosine deaminase AID, nCas9 nuclease and uracil DNA glycosidase.

In the d4), the coding gene of the cytosine deaminase AID and the coding gene of the nCas9 nuclease are introduced into the receptor organism or the cells of the receptor organism through a recombinant plasmid B; the recombinant plasmid B expresses a fusion protein composed of cytosine deaminase AID and nCas9 nuclease.

In a specific embodiment of the present invention, the nucleotide sequence of the recombinant plasmid B is as shown in SEQ ID NO: 1; the nucleotide sequence of the recombinant plasmid A is as shown in SEQ ID NO: 3.

In the above method for mutating a target base C to A in a genome sequence, the receptor organism can be prokaryotes; the C-to-A mutation is a process of base mutating C to A in prokaryotes.

Further, the prokaryote can be Escherichia coli.

Even further, the Escherichia coli is specifically wild Escherichia coli MG1655 or Escherichia coli ATCC 8739.

In order to realize the above objectives, the present invention also provides a method for improving a base editing efficiency of mutating a target base C to A in a genome sequence.

The method for improving a base editing efficiency of mutating a target base C to A in a genome sequence provided by the present invention can be D1) or D3) as described above or d1) or d3) as described above.

In order to realize the above objectives, the present invention also provides a method for improving a base editing efficiency of mutating a target base C to G in a genome sequence.

The method for improving a base editing efficiency of mutating a target base C to G in a genome sequence provided by the present invention can be E1) or E2) as follows:

E1) the method includes the following steps: using a CRISPR/Cas9 system, cytosine deaminase and uracil DNA glycosidase for single-base editing to improve a base editing efficiency of mutating a target base C to G;

E2) the method includes the following steps: using a CRISPR/Cas9 system, cytosine deaminase APOBEC and uracil DNA glycosidase for single-base editing to improve a base editing efficiency of mutating a target base C to G.

Further, the above method for improving a base editing efficiency of mutating a target base C to G in a genome sequence can be e1) or e2) as follows:

e1) the method includes the following steps: introducing a coding gene of cytosine deaminase, a coding gene of CRISPR nuclease, a coding gene of uracil DNA glycosidase and a coding sequence of sgRNA into a receptor organism or the cells of a receptor organism, so that the coding gene of cytosine deaminase, the coding gene of CRISPR nuclease, the coding gene of uracil DNA glycosidase and the coding sequence of sgRNA are all expressed to improve a base editing efficiency of mutating a target base C to G in a genome sequence;

e2) the method includes the following steps: introducing a coding gene of cytosine deaminase APOBEC, a coding gene of nCas9 nuclease, a coding gene of uracil DNA glycosidase and a coding sequence of sgRNA into a receptor organism or the cells of a receptor organism, so that the coding gene of cytosine deaminase APOBEC, the coding gene of nCas9 nuclease, the coding gene of uracil DNA glycosidase and the coding sequence of sgRNA are all expressed to improve a base editing efficiency of mutating a target base C to G in a genome sequence;

The sgRNA targets a target sequence, and the target base locates in the target sequence.

In e1) and e2), the cytosine deaminase or the cytosine deaminase APOBEC, the CRISPR nuclease or the nCas9 nuclease can be subject to fusion expression or free expression with the uracil DNA glycosidase in the receptor organism or the cells of the receptor organism.

The cytosine deaminase can be cytosine deaminase from different sources, such as mouse-derived cytosine deaminase APOBEC1 (GenBank: AAH03792.1), human-derived cytosine deaminase APOBEC3A (GenBank: AK3E3285.1), lamprey-derived cytosine deaminase pmCDA (Accession: ABO15149.1), etc. Specifically, the cytosine deaminase or the cytosine deaminase APOBEC is mouse-derived cytosine deaminase APOBEC1, and its amino acid sequence is well known in the art; in an example of the present invention, an amino acid sequence of the cytosine deaminase used is as shown in Accession: AAH03792.1 of NCBI.

The uracil DNA glycosidase can be uracil DNA glycosidase from different sources, such as human-derived uracil DNA glycosidase UNG (GenBank: CAG46474.1), yeast-derived uracil DNA glycosidase ungl (Accession: CAA86634.1), Escherichia coli-derived uracil DNA glycosidase ung (Accession: EGT65982.1), etc. Specifically, the uracil DNA glycosidase is modified human-derived uracil DNA glycosidase UNG, and its amino acid sequence is obtained by deleting an amino acid sequence shown at sites 1-84 from the N-terminal of an amino acid sequence of human-derived uracil DNA glycosidase UNG as shown in GenBank: CAG46474.1 of NCBI.

The CRISPR nuclease can be CRISPR nuclease from different sources or a mutant thereof, such as Streptococcus pyogenes derived Cas9 nuclease (Accession: Q99ZW2.1) or a mutant thereof, Staphylococcus aureus derived Cas9 nuclease (Accession: AYD60528.1) or a mutant thereof, Francisella tularensis derived cpf1 nuclease (Accession: A0Q7Q2.1) or a mutant thereof. Specifically, the CRISPR nuclease or the nCas9 nuclease is a mutant nCas9-D10A of Cas9, and its amino acid sequence is well known in the art. In an example of the present invention, an amino acid sequence of the CRISPR nuclease used is an amino acid sequence obtained by mutating aspartic acid (D) to alanine (A) at a site 10 from an N-terminal of an amino acid sequence as shown in Accession: Q99ZW2.1 of NCBI.

Even further, in e2), the coding gene of the cytosine deaminase APOBEC, the coding gene of the nCas9 nuclease and the coding gene of the uracil DNA glycosidase are introduced into the receptor organism or the cells of the receptor organism through a recombinant plasmid C.

The recombinant plasmid C expresses a fusion protein composed of cytosine deaminase APOBEC, nCas9 nuclease and uracil DNA glycosidase.

In a specific embodiment of the present invention, the nucleotide sequence of the recombinant plasmid C is as shown in SEQ ID NO: 5.

In the method for improving a base editing efficiency of mutating a target base C to G in a genome sequence, the receptor biological cells can be eukaryotic cells.

Further, the eukaryotic cells can be mammalian cells. The mammal includes human.

Even further, the mammalian cells are specifically HEK293T cells or Hela cells.

In order to realize the above objectives, the present invention also provides a method for realizing a site-directed mutation from any base to any base in a genome sequence in prokaryotes.

The method (its principle is as shown in FIG. 1) for realizing a site-directed mutation from any base to any base in a genome sequence in prokaryotes can be M1) or M2) or M3) or M4) as follows:

M1 includes m1) or m2) or m3):

m1) when a target base in a genome sequence is a base C, starting from the base C, the target base can be mutated from the base C to the base T using a base editing system that mutate C to T, so as to realize the editing from the base C to the base T;

m2) when a target base in a genome sequence is a base C, starting from the base C, the target base can be mutated from the base C to a base A using a base editing system for mutating C to A so as to realize the editing from the base C to the base A;

m3) when a target base in a genome sequence is a base C, a mutant taking a base A as the target base is obtained according to the method described in m2); starting from the base A, the target base can be mutated from the base A to a base G using a base editing system for mutating A to G, so as to realize the editing from the base C to the base G;

any site-directed mutation from the base C to the base T, the base A and the base G is therefore realized;

M2) when a target base in a genome sequence is a base G, since the base G is a complementary base of a base C, any site-directed mutation from the base G to the base A, the base T and the base C is also realized according to the method described in M1);

M3 includes m4) or m5) or m6):

m4) when a target base in a genome sequence is a base T, a base A is a complementary base of the target base; starting from the base A, the complementary base of the target base can be mutated from the base A to a base G using a base editing system for mutating A to G so as to realize the editing from the base T to the base G;

m5) when a target base in a genome sequence is a base T, a mutated base C is obtained according to the method described in m4); starting from the base C, the target base can be mutated from the base C to a base A using a base editing system for mutating C to A so as to realize the editing from the base T to the base A;

m6) when a target base in a genome sequence is a base T, amutated base A is obtained according to the method described in m5); starting from the base A, the target base can be mutated from the base A to a base G using a base editing system for mutating A to G so as to realize the editing from the base T to the base G;

any site-directed mutation from the base T to the base C, the base A and the base G is therefore realized;

M4) when a target base in a genome sequence is a base A, since the base A is a complementary base of a base T, any site-directed mutation from the base A to the base G, the base T and the base C is also realized according to the method described in M3);

The base editing system for mutating C to A is a base editing system I for mutating C to A, or a base editing system II for mutating C to A, or a base editing system III for mutating C to A, or a base editing system IV for mutating C to A;

The base editing system I for mutating C to A includes cytosine deaminase or a biomaterial related to the cytosine deaminase, CRISPR nuclease or a biomaterial related to the CRISPR nuclease, and uracil DNA glycosidase or a biomaterial related to the uracil DNA glycosidase;

The base editing system II for mutating C to A includes cytosine deaminase or a biomaterial related to the cytosine deaminase, and CRISPR nuclease or a biomaterial related to the CRISPR nuclease;

The base base editing system III for mutating C to A includes cytosine deaminase AID or a biomaterial related to the cytosine deaminase AID, nCas9 nuclease or a biomaterial related to the nCas9 nuclease and uracil DNA glycosidase or a biomaterial related to the uracil DNA glycosidase;

The base editing system IV for mutating C to A includes cytosine deaminase AID or a biomaterial related to the cytosine deaminase AID, and nCas9 nuclease or a biomaterial related to the nCas9 nuclease.

Further, the cytosine deaminase can be cytosine deaminase from different sources, such as mouse-derived cytosine deaminase APOBEC1 (GenBank: AAH03792.1), human-derived cytosine deaminase APOBEC3A (GenBank: AKE33285.1), lamprey-derived cytosine deaminase pmCDA (Accession: ABO15149.1), etc. Specifically, the cytosine deaminase or the cytosine deaminase AID is lamprey-derived cytosine deaminase pmCDA, and its amino acid sequence is well known in the art; in an example of the present invention, an amino acid sequence of the cytosine deaminase used is as shown in Accession: ABO15149.1 of NCBI.

The uracil DNA glycosidase can be uracil DNA glycosidase from different sources, such as human-derived uracil DNA glycosidase UNG (GenBank: CAG46474.1), yeast-derived uracil DNA glycosidase ungl (Accession: CAA86634.1), Escherichia coli-derived uracil DNA glycosidase ung (Accession: EGT65982.1), etc. Specifically, the uracil DNA glycosidase is Escherichia coli-derived uracil DNA glycosidase ung, and its amino acid sequence is well known in the art; in an example of the present invention, an amino acid sequence of the Escherichia coli-derived uracil DNA glycosidase used is as shown in Accession: EGT65982.1 of NCBI.

The CRISPR nuclease can be CRISPR nuclease from different sources or a mutant thereof, such as Streptococcus pyogenes derived Cas9 nuclease (Accession: Q99ZW2.1) or a mutant thereof, Staphylococcus aureus derived Cas9 nuclease (Accession: AYD60528.1) or a mutant thereof, Francisella tularensis derived cpf1 nuclease (Accession: A0Q7Q2.1) or a mutant thereof. Specifically, the CRISPR nuclease or the nCas9 nuclease is a mutant nCas9-D10A of Cas9, and its amino acid sequence is well known in the art. In an example of the present invention, an amino acid sequence of the CRISPR nuclease used is an amino acid sequence obtained by mutating aspartic acid (D) to alanine (A) at a site 10 from the N-terminal of an amino acid sequence as shown in Accession: Q99ZW2.1 of NCBI.

The prokaryote is Escherichia coli.

Even further, the Escherichia coli is Escherichia coli MG1655 or Escherichia coli ATCC 8739.

In order to realize the above objectives, the present invention also provides a method for realizing a site-directed mutation from any base to any base in a genome sequence in eukaryotes.

The method (its principle is as shown in FIG. 2) for realizing a site-directed mutation from any base to any base in a genome sequence in eukaryotes can be N1) or N2) or N3) or N4) as follows:

N1) includes n1) or n2) or n3):

n1) when a target base in a genome sequence is a base C, starting from the base C, the target base can be mutated from the base C to a base T using a base editing system for mutating C to T to realize the base editing from C to T;

n2) when a target base in a genome sequence is a base C, starting from the base C, the target base can be mutated from the base C to a base G using a base editing system for mutating C to G so as to realize the editing from the base C to the base G;

n3) when a target base in a genome sequence is a base C, a mutant taking a base G as the target base is obtained according to the method described in n2), and the base C is a complementary base of the base G; starting from the base C, the target base can be mutated from the base C to a base T using a base editing system for mutating C to T, and a base A is a complementary base of the base T, realizing the editing from the base C to the base A;

any site-directed mutation from the base C to the base T, the base A and the base G is therefore realized;

N2) when a target base in a genome sequence is a base G, since the base G is a complementary base of a base C, any site-directed mutation from the base G to the base A, the base T and the base C is also realized according to the method described in N1);

N3 includes n4) or n5) or n6):

n4) when a target base in a genome sequence is a base T, a base A is a complementary base of the base T; starting from the base A, the complementary base of the target base can be mutated from the base A to a base G using a base editing system for mutating A to G so as to realize the editing from the base T to the base G;

n5) when a target base in a genome sequence is a base T, a mutated C is obtained according to the method described in n4); starting from the base C, the target base can be mutated from the base C to a base G using a base editing system for mutating C to G so as to realize the editing from the base T to the base G;

n6) when a target base in a genome sequence is a base T, a mutated base G is obtained according to the method described in n5), and a base C is a complementary base of the base G; starting from the base C, the complementary base of the target base can be mutated from the base C to a base T using a base editing system for mutating C to T so as to realize the editing from the base T to the base A;

any site-directed mutation from the base T to the base C, the base A and the base G is therefore realized;

N4) when a target base in a genome sequence is a base A, since the base A is a complementary base of a base T, any site-directed mutation from the base A to the base G, the base T and the base C is also realized according to the method described in N3);

The base editing system for mutating C to G is a base editing system I for mutating C to G, or a base editing system II for mutating C to G, or a base editing system III for mutating C to G, or a base editing system IV for mutating C to G;

The base editing system I for mutating C to G includes cytosine deaminase or a biomaterial related to the cytosine deaminase, CRISPR nuclease or a biomaterial related to the CRISPR nuclease, and uracil DNA glycosidase or a biomaterial related to the uracil DNA glycosidase;

The base editing system II for mutating C to G includes cytosine deaminase or a biomaterial related to the cytosine deaminase, and CRISPR nuclease or a biomaterial related to the CRISPR nuclease;

The base editing system III for mutating C to G includes cytosine deaminase APOBEC or a biomaterial related to the cytosine deaminase APOBEC, nCas9 nuclease or a biomaterial related to the nCas9 nuclease and uracil DNA glycosidase or a biomaterial related to the uracil DNA glycosidase;

The base editing system IV for mutating C to G includes cytosine deaminase APOBEC or a biomaterial related to the cytosine deaminase APOBEC, and nCas9 nuclease or a biomaterial related to the nCas9 nuclease.

Further, the cytosine deaminase can be cytosine deaminase from different sources, such as mouse-derived cytosine deaminase APOBEC1 (GenBank: AAH03792.1), human-derived cytosine deaminase APOBEC3A (GenBank: AKE33285.1), lamprey-derived cytosine deaminase pmCDA (Accession: ABO15149.1), etc. Specifically, the cytosine deaminase or the cytosine deaminase APOBEC is mouse-derived cytosine deaminase APOBEC1, and its amino acid sequence is well known in the art; in an example of the present invention, an amino acid sequence of the cytosine deaminase used is as shown in Accession: AAH03792.1 of NCBI.

The uracil DNA glycosidase can be uracil DNA glycosidase from different sources, such as human-derived uracil DNA glycosidase UNG (GenBank: CAG46474.1), yeast-derived uracil DNA glycosidase ungl (Accession: CAA86634.1), Escherichia coli-derived uracil DNA glycosidase ung (Accession: EGT65982.1), etc. Specifically, the uracil DNA glycosidase is modified human-derived uracil DNA glycosidase UNG, and its amino acid sequence is obtained by deleting the amino acid sequence of 1-84 from the human-derived uracil DNA glycosidase UNG, as shown in GenBank: CAG46474.1 of NCBI.

The CRISPR nuclease can be CRISPR nuclease from different sources or a mutant thereof, such as Streptococcus pyogenes derived Cas9 nuclease (Accession: Q99ZW2.1) or a mutant thereof, Staphylococcus aureus derived Cas9 nuclease (Accession: AYD60528.1) or a mutant thereof, Francisella tularensis derived cpf1 nuclease (Accession: A0Q7Q2.1) or a mutant thereof. Specifically, the CRISPR nuclease or the nCas9 nuclease is a mutant nCas9-D10A of Cas9, and its amino acid sequence is well known in the art. In an example of the present invention, an amino acid sequence of the CRISPR nuclease used is an amino acid sequence obtained by mutating aspartic acid (D) to alanine (A) at a site 10 from the N-terminal of an amino acid sequence as shown in Accession: Q99ZW2.1 of NCBI.

The eukaryotes are eukaryotic cells.

Even further, the eukaryotic cells are mammalian cells, such as HEK293T cells or Hela cells.

In order to realize the above objective, the present invention provides any of the following applications described in a1)-a8):

a1) an application of the base editing system I for mutating C to A in mutating a target base C to A in a genome sequence;

a2) an application of the C to A base editing system II in mutating a target base C to A in a genome sequence;

a3) an application of uracil DNA glycosidase or the base editing system I for mutating C to A in improving a base editing efficiency of mutating a target base C to A in a genome sequence;

a4) an application of uracil DNA glycosidase or the base editing system I for mutating C to G in improving a base editing efficiency of mutating a target base C to G in a genome sequence;

a5) an application of the base editing system I for mutating C to A, a base editing system for mutating C to T and a base editing system for mutating A to G in realizing a site-directed mutation from any base to any base in a genome sequence in prokaryotes;

a6) an application of the base editing system II for mutating C to A, a base editing system for mutating C to T and a base editing system for mutating A to G in realizing a site-directed mutation from any base to any base in a genome sequence in prokaryotes;

a7) an application of the base editing system I for mutating C to G, a base editing system for mutating C to T and a base editing system for mutating A to G in realizing a site-directed mutation from any base to any base in a genome sequence in eukaryotes; and

a8) an application of the base editing system II for mutating C to G, a base editing system for mutating C to T and a base editing system for mutating A to G in realizing a site-directed mutation from any base to any base in a genome sequence in eukaryotes.

In order to realize the above objective, the present invention provides any of the following applications described in b1)-b8):

b1) an application of the base base editing system III for mutating C to A in mutating a target base C to A in a genome sequence;

b2) an application of the base editing system IV for mutating C to A in mutating a target base C to A in a genome sequence;

b3) an application of uracil DNA glycosidase or the base base editing system III for mutating C to A in improving a base editing efficiency of mutating a target base C to A in a genome sequence;

b4) an application of uracil DNA glycosidase or the base editing system III for mutating C to G in improving a base editing efficiency of mutating a target base C to G in a genome sequence;

b5) an application of the base base editing system III for mutating C to A, a base editing system for mutating C to T and a base editing system for mutating A to G in realizing a site-directed mutation from any base to any base in a genome sequence in prokaryotes;

b6) an application of the base editing system IV for mutating C to A, a base editing system for mutating C to T and a base editing system for mutating A to G in realizing a site-directed mutation from any base to any base in a genome sequence in prokaryotes;

b7) an application of the base editing system III for mutating C to G, a base editing system for mutating C to T and a base editing system for mutating A to G in realizing a site-directed mutation from any base to any base in a genome sequence in eukaryotes; and

b8) an application of the base editing system IV for mutating C to G, a base editing system for mutating C to T and a base editing system for mutating A to G in realizing a site-directed mutation from any base to any base in a genome sequence in eukaryotes.

In order to realize the above objective, the present invention provides any of the following products described in c1)-c5):

c1) a product for mutating a target base C to A in a genome sequence, including the base editing system I for mutating C to A, or the base editing system II for mutating C to A, or the base base editing system III for mutating C to A, or the base editing system IV for mutating C to A;

c2) a product for improving a base editing efficiency of mutating a target base C to A in a genome sequence, including the base editing system I for mutating C to A, or the base base editing system III for mutating C to A;

c3) a product for improving a base editing efficiency of mutating a target base C to G in a genome sequence, including the base editing system I for mutating C to G, or the base editing system III for mutating C to G;

c4) a product for realizing a site-directed mutation from any base to any base in a genome sequence in prokaryotes, including a base editing system for mutating C to A, a base editing system for mutating C to T, and a base editing system for mutating A to G; wherein the base editing system for mutating C to A is the base editing system I for mutating C to A, or the base editing system II for mutating C to A, or the base base editing system III for mutating C to A, or the base editing system IV for mutating C to A; and

c5) a product for realizing a site-directed mutation from any base to any base in a genome sequence in eukaryotes, including a base editing system for mutating C to G, a base editing system for mutating C to T, and a base editing system for mutating A to G; wherein the base editing system for mutating C to G is the base editing system I for mutating C to G, or the base editing system II for mutating C to G, or the base editing system III for mutating C to G, or the base editing system IV for mutating C to G.

In any of the applications or products or methods described above, in the a1) or a2) or a3) or b1) or b2) or b3) or c1) or c2) or d1) or d2) or d3) or d4), the mutation of a target base C to A is mutating a target base C to A in prokaryotes.

In the a4) or b4) or c3) or e1) or e2), the mutation of a target base C to G is mutating a target base C to G in eukaryotes.

In any of the applications or products described above, the cytosine deaminase can be cytosine deaminase from different sources, such as mouse-derived cytosine deaminase APOBEC1 (GenBank: AAH03792.1), human-derived cytosine deaminase APOBEC3A (GenBank: AKE33285.1), and lamprey-derived cytosine deaminase pmCDA (Accession: ABO15150.1). Specifically, the cytosine deaminase used in the C-to-A base editing in prokaryotes (such as Escherichia coli) is lamprey-derived cytosine deaminase pmCDA, and its amino acid sequence is well known in the art; in an example of the present invention, an amino acid sequence of the cytosine deaminase used is as shown in Accession: ABO15149.1 of NCBI; the cytosine deaminase used in the C-to-G base editing in an eucaryo (such as mammalian cells) is mouse-derived cytosine deaminase APOBEC1, and its amino acid sequence is well known in the art; in an example of the present invention, an amino acid sequence of the cytosine deaminase used is as shown in Accession: AAH03792.1 of NCBI.

The cytosine deaminase or the cytosine deaminase AID or the cytosine deaminase APOBEC is any of X1)-X5) as follows:

X1) a nucleic acid molecule of the cytosine deaminase or the cytosine deaminase AID or the cytosine deaminase APOBEC;

X2) an expression cassette containing the nucleic acid molecule described in X1);

X3) a recombinant vector of the nucleic acid molecule described in X1), or a recombinant vector containing the expression cassette described in X2);

X4) a recombinant microorganisim of the nucleic acid molecule described in X1), or a recombinant microorganisim containing the expression cassette described in X2), or a recombinant microorganisim containing the recombinant vector described in X3); and

X5) a transgenic cell line of the nucleic acid molecule described in X1), or a transgenic cell line containing the expression cassette described in X2).

The nucleic acid molecule for encoding the cytosine deaminase is x1) or x2) or x3) as follows:

x1) a cDNA molecule or DNA molecule as shown at sites 4,405-5,028 (for encoding cytosine deaminase pmCDA) of SEQ ID NO: 1 or at sites 1,038-1,721 (for encoding cytosine deaminase APOBEC1) of SEQ ID NO: 4 in a sequence list;

x2) a cDNA molecule or DNA molecule which has 75% or higher identity with a nucleotide sequence confined by xl) and encodes the cytosine deaminase; and

x3) a cDNA molecule or DNA molecule which hybridizes with a nucleotide sequence confined by x1) or x2) under a strict condition and encodes the cytosine deaminase.

The CRISPR nuclease can be CRISPR nuclease from different sources or a mutant thereof, such as Streptococcus pyogenes derived Cas9 nuclease (Accession: Q99ZW2.1) or a mutant thereof, Staphylococcus aureus derived Cas9 nuclease (Accession: AYD60528.1) or a mutant thereof, Francisella tularensis derived cpf1 nuclease (Accession: A0Q7Q2.1) or a mutant thereof. Specifically, the CRISPR nuclease used in the C-to-A base editing in prokaryotes (such as Escherichia coli) or in the C-to-G base editing in an eucaryotes (such as mammalian cells) is a mutant nCas9-D10A of Streptococcus pyogenes derived Cas9, and its amino acid sequence is well known in the art. In an example of the present invention, an amino acid sequence of the CRISPR nuclease used is an amino acid sequence obtained by mutating aspartic acid (D) to alanine (A) at a site 10 from the N-terminal of an amino acid sequence as shown in Accession: Q99ZW2.1 of NCBI.

A biomaterial related to the CRISPR nuclease or the nCas9 nuclease is any of the following described in Y1)-Y5):

Y1) a nucleic acid molecule for encoding the CRISPR nuclease or the nCas9 nuclease;

Y2) an expression cassette containing the nucleic acid molecule described in Y1);

Y3) a recombinant vector containing the nucleic acid molecule described in Y1), or a recombinant vector containing the expression cassette described in Y2);

Y4) a recombinant microorganisim of the nucleic acid molecule described in Y1), or a recombinant microorganisim containing the expression cassette described in Y2), or a recombinant microorganisim containing the recombinant vector described in Y3); and

Y5) a transgenic cell line of the nucleic acid molecule described in Y1), or a transgenic cell line containing the expression cassette described in Y2).

The nucleic acid molecule for encoding the Cas9 mutant nCas9-D10A is y1) or y2) or y3) as follows:

y1) a cDNA molecule or DNA molecule as shown at sites 1-4,104 of SEQ ID NO: 1 in a sequence list;

y2) a cDNA molecule or DNA molecule which has 75% or higher identity with a nucleotide sequence confined by y1) and encodes the Cas9 nuclease; and

y3) a cDNA molecule or DNA molecule which hybridizes with a nucleotide sequence confined by y1) or y2) under a strict condition and encodes the Cas9 nuclease.

The uracil DNA glycosidase can be uracil DNA glycosidase from different sources, such as human-derived uracil DNA glycosidase UNG (GenBank: CAG46474.1), yeast-derived uracil DNA glycosidase ung1 (Accession: CAA86634.1), Escherichia coli-derived uracil DNA glycosidase ung (Accession: EGT65982.1), etc. Specifically, the uracil DNA glycosidase used in the C-to-A base editing in prokaryotes (such as Escherichia coli) is Escherichia coli-derived uracil DNA glycosidase ung, and its amino acid sequence is well known in the art; in an example of the present invention, an amino acid sequence of the uracil DNA glycosidase used is as shown in Accession: EGT65982.1 of NCBI; the uracil DNA glycosidase used in the C-to-G base editing in an eucaryo (such as mammalian cells) is modified human-derived uracil DNA glycosidase UNG, and its amino acid sequence is obtained by deleting an amino acid sequence shown at sites 1-84 from the N-terminal of an amino acid sequence of human-derived uracil DNA glycosidase UNG as shown in GenBank: CAG46474.1 of NCBI.

A biomaterial related to the CRISPR nuclease or the nCas9 nuclease is any of the following described in Z1)-Z5):

Z1) a nucleic acid molecule for encoding the uracil DNA glycosidase;

Z2) an expression cassette containing the nucleic acid molecule described in Z1);

Z3) a recombinant vector of the nucleic acid molecule described in Z1), or a recombinant vector containing the expression cassette described in Z2);

Z4) a recombinant microorganisim of the nucleic acid molecule described in Z1), or a recombinant microorganisim containing the expression cassette described in Z2), or a recombinant microorganisim containing the recombinant vector described in Z3); and

Z5) a transgenic cell line of the nucleic acid molecule described in Z1), or a transgenic cell line containing the expression cassette described in Z2).

The nucleic acid molecule for encoding the uracil DNA glycosidase is z1) or z2) or z3) as follows:

z1) a cDNA molecule or DNA molecule as shown at sites 1-687 (for encoding Escherichia coli-derived uracil DNA glycosidase ung) of SEQ ID NO: 3 or at sites 1-663 (for encoding modified human-derived uracil DNA glycosidase UNG) of SEQ ID NO: 5 in a sequence list;

z2) a cDNA molecule or DNA molecule which has 75% or higher identity with a nucleotide sequence confined by z1) and encodes the uracil DNA glycosidase; and

z3) a cDNA molecule or DNA molecule which hybridizes with a nucleotide sequence confined by z1) or z2) under a strict condition and encodes the uracil DNA glycosidase.

In any of the methods or applications or products, the base editing system for mutating C to T can be any base editing system capable of mutating C to T in the prior art well known to those skilled in the art, such as a base editing system including cytosine deaminase (cytosine deaminase APOBEC1) and nCas9 nuclease, or a base editing system including cytosine deaminase (cytosine deaminase APOBEC1), nCas9 nuclease and uracil DNA glycosylase inhibitory protein UGI.

In any of the methods or applications or products, the base editing system for mutating A to G can be any base editing system capable of mutating C to T in the prior art well known to those skilled in the art, such as a base editing system including adenine deaminase (adenine deaminase TadA) and nCas9 nuclease.

In any of the methods or products, the base editing system also includes sgRNA; the sgRNA targets a target sequence, and the target base locates in the target sequence.

In any of the methods or applications or products, the any base is A, G, C or T.

### BRIEF DESCRIPTION OF THE DRAWINGS

FIG. 1 shows schematic diagrams of the base editing for mutating A, T, C or G to any base by combining a base editing system for mutating C to A, a base editing system for mutating C to T, and a base editing system for mutating A to G. The upper figure shows a schematic diagram of the base editing for mutating any base starting from C or G; and the lower figure shows a schematic diagram of the base editing for mutating any base starting from A or T.

FIG. 2 shows schematic diagrams of the base editing for mutating A, T, C or G to any base by combining a base editing system for mutating C to G, a base editing system for mutating C to T and a base editing system for mutating A to G. The upper figure shows a schematic diagram of the base editing for mutating any base starting from C or G; and the lower figure shows a schematic diagram of the base editing for mutating any base starting from A or T.

FIG. 3 is a map of the ptrc_nCas9_AID plasmid (pnCas9_AID plasmid).

FIG. 4 is a map of the Escherichia coli gRNA plasmid.

FIG. 5 is a map of the ptrc_ung_nCas9_AID plasmid (pUNG_nCas9_AID plasmid).

FIG. 6 is a map of the pAPOBEC_nCas9 plasmid (pAPOBEC_nCas9_UGI plasmid).

FIG. 7 is a map of the pAPOBEC_nCas9_UNG plasmid.

FIG. 8 is a map of the mammalian cells gRNA plasmid.

FIGS. 9A and 9B shows target genes, target sequences and editing results of example 3. FIG. 9A shows target genes, target sequences and editing results of base-directed replacement in HEK293T cells; FIG. 9B shows target genes, target sequences and editing results of base-directed replacement in Hela cells.

FIG. 10 is a map of the pTadA_nCas9 plasmid.

FIGS. 11A and 11B shows target genes, target sequences and editing results of example 4. FIG. 11A shows an editing efficiency of mutating a base C to any base; FIG. 11B shows an editing efficiency of mutating a base T to any base.

FIG. 12 is a map of the xcas9 (3.7)-ABE (7.10) plasmid.

FIG. 13 shows target genes, target sequences and editing results of example 5.

### DETAILED DESCRIPTION OF THE EMBODIMENTS

The following embodiments are intended for a better understanding of the present invention, but not limiting the present invention. Unless otherwise noted, all the experimental methods in the following embodiments are conventional methods. Unless otherwise noted, all the experimental materials in the following embodiments can be purchased from conventional biochemical reagent shops. Three repeated experiments are set for the quantitative tests in the following embodiments, and a mean value is calculated as a result.

In the following embodiments, HEK293T cells, Hela cells, wild Escherichia coli MG1655, and wild Escherichia coli ATCC 8739 are all products of the American Type Culture Collection (ATCC).

In the following embodiments, the cytosine deaminase APOBEC used in mammalian cells is cytosine deaminase APOBEC1 (GenBank: AAH03792.1), and its encoding gene sequence is as shown at sites 1,038-1,721 in SEQ ID NO: 4.

In the following embodiments, the cytosine deaminase AID used in Escherichia coli is cytosine deaminase pmCDA (GenBank: ABO15149.1), and its encoding gene sequence is as shown at sites 4,405-5,028 in SEQ ID NO: 1.

In the following embodiments, the uracil DNA glycosidase used in Escherichia coli is Escherichia coil-derived uracil DNA glycosidase ung (GenBank: EGT65982.1), and its encoding gene sequence is as shown at sites 1-687 in SEQ ID NO: 3.

In the following embodiments, the uracil DNA glycosidase used in mammalian cells is modified human-derived uracil DNA glycosidase UNG, and its amino acid sequence is obtained by deleting an amino acid sequence shown at sites 1-84 from an amino acid sequence of human-derived uracil DNA glycosidase UNG (GenBank: CAG46474.1); its encoding gene sequence is as shown at sites 1-663 in SEQ ID NO: 5.

In the following embodiments, the nCas9 nuclease used in mammalian cells and Escherichia coli is a mutant nCas9-D10A of Cas9, and its amino acid sequence is obtained by mutating aspartic acid (D) to alanine (A) at a site 10 from the N -terminal of an amino acid sequence of the Cas9 nuclease (Accession: Q99ZW2.1); its encoding gene sequence is as shown at sites 1-4,104 in SEQ ID NO: 1.

In the following embodiments, the PCR detection primer sequences after editing a target sequence of genes are as shown in Table 1.

TABLE 1
PCR Detection Primer Sequences
After Editing a Target Sequence of
Genes

| Primer Name | Primer Sequence |
|---|---|
| dcuA_genome_F | TGCTGGCGATCTTCTTGGG |
| | (SEQ ID NO: 8) |
| dcuA_genome_R | CCCGTGTCATCCATCTGTACC |
| | (SEQ ID NO: 9) |
| dcuB_genome_F | AACGGATCGCTGGTTATCTG |
| | (SEQ ID NO: 10) |
| dcuB_genome_R | CCGGTACGGAGATGAATTTCTG |
| | (SEQ ID NO: 11) |
| dcuC_genome_F | ATCGGCGCGAATGATATG |
| | (SEQ ID NO: 12) |
| dcuC_genome_R | ATCACTAGCCCAACAAGC |
| | (SEQ ID NO: 13) |
| dcuD_genome_F | CGGTTATGCCCGCTACATGG |
| | (SEQ ID NO: 14) |
| dcuD_genome_R | GGGATCGCTGTTCGCTTCAC |
| | (SEQ ID NO: 15) |
| relA_genome_F | TCGCGTACTGGATCTGTTCTGC |
| | (SEQ ID NO: 16) |
| relA_genome_R | GTTGCCAACACCTTCGACTACC |
| | (SEQ ID NO: 17) |
| rpoS_genome_F | AACCAGTACGCCTATCTC |
| | (SEQ ID NO: 18) |
| rpo SgenomeR | ACTCAGGGTTCTGGATTG |
| | (SEQ ID NO: 19) |
| spoT_genome_F | CCTGGCCTTTGAGATGAG |
| | (SEQ ID NO: 20) |
| spoT_genome_R | GTTCAGGACGCTGTAGAG |
| | (SEQ ID NO: 21) |
| lacZ1_genome_F | AGTTGCGTGACTACCTAC |
| | (SEQ ID NO: 22) |
| lacZ1_genome_R | AGACCAGACCGTTCATAC |
| | (SEQ ID NO: 23) |
| lacZ2_genome_F | CGTCTGAATTTGACCTGAG |
| | (SEQ ID NO: 24) |
| lacZ2_genome_R | CCGTCGATATTCAGCCATGTG |
| | (SEQ ID NO: 25) |
| ung_genome_F | CCCTCTTCCGCTTAGTAACTTG |
| | (SEQ ID NO: 26) |
| ung_genome_R | GAAGTGTTGCGTCGTCAG |
| | (SEQ ID NO: 27) |
| RNF2_genome_F | CCTGATCACCTCCCAAAGTC |
| | (SEQ ID NO: 28) |
| RNF2_genome_R | CCTGATCACCTCCCAAAGTC |
| | (SEQ ID NO: 29) |

### EXAMPLE 1

### A Base Editing Method of Mutating C to A in Escherichia coli

The fusion expression of cytosine deaminase (AID) and nCas9 in Escherichia coli can achieve a site-directed mutation of cytosine (C) to thymine (T) as well as cytosine (C) to adenine (A) at a specific site of Escherichia coli under the guidance of gRNA; wherein C-to-T mutations accounted for 40.7% of total mutations and C-to-A mutations accounted for 59.3% of the total mutations.

### I. Test Methods

The pnCas9_AID plasmid containing a cytosine deaminase (AID) and nCas9 fusion expression system and an Escherichia coli gRNA plasmid containing different target sites were introduced into wild Escherichia coli MG1655 or wild Escherichia coli ATCC 8739, plating was performed after culturing for 24 h, and a part of colonies were randomly selected for PCR detection and sequencing of an edited site.

A map of the pnCas9_AID plasmid was shown in FIG. 3, and its nucleotide sequence was as shown in SEQ ID NO:1; wherein sites 1-4,104 represented an encoding gene sequence of the nCas9, sites 4,405-5,028 represented an encoding gene sequence of the cytosine deaminase (AID), sites 6,609-7,268 were chloramphenicol genes, and sites 8,335-6,245 were origins of replication. The pnCas9_AID plasmid expressed a fusion protein composed of cytosine deaminase (AID) and nCas9.

The Escherichia coli gRNA plasmid containing different target sites was a gRNA plasmid targeting at different genes such as dcuA, dcuB, dcuC, dcuD, relA, rpoS and lacZ or targeting at different sites of the same gene. The specific target sites of the gRNA plasmid were as shown in Table 2.

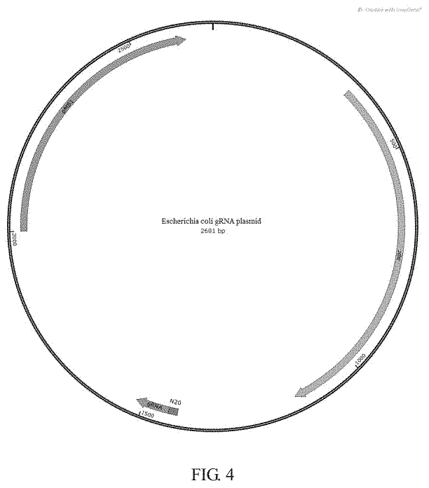

Taking the gRNA plasmid targeting at sites 1,444-1,463 of a lacZ gene as an example, its map was shown in FIG. 4, and its nucleotide sequence was as shown in SEQ ID NO: 2; wherein sites 336-1,148 were apramycin genes, sites 1,421-1,440 represented the target sequence sites 1,441-1,518 represented a gRNA sequence, and sites 2,001-2,620 were origins of replication. In this example or hereinafter, the Escherichia coli gRNA plasmids targeting at other sites can be obtained simply by replacing the target sequence in the gRNA plasmid shown in SEQ ID NO: 2 by the target sequence of other genes or other target sequences of the same gene.

TABLE 2
Specific Target Sites of the gRNA Plasmid

| | N | N | N | N | N | N | N | N | N | N | N | N | N | N | N | N | N | N | N | N | E.coli | N | N | N | N | N | N | N | N | N | N | N | | | | | | | | N | |
|---|---|---|---|---|---|---|---|---|---|---|---|---|---|---|---|---|---|---|---|---|---|---|---|---|---|---|---|---|---|---|---|---|---|---|---|---|---|---|---|---|---|
| E.coli ATCC8739 | 20 | 19 | 18 | 17 | 16 | 15 | 14 | 13 | 12 | 11 | 10 | 9 | 8 | 7 | 6 | 5 | 4 | 3 | 2 | 1 | MG1655 | 20 | 19 | 18 | 17 | 16 | 15 | 14 | 13 | 12 | 11 | 10 | N9 | N8 | N7 | N6 | N5 | N4 | N3 | 2 | N1 |
| dcuA643-662 wt | A | A | G | C | A | G | A | T | T | G | A | A | A | T | C | A | A | A | T | C | lacZ 1444- | T | T | A | C | G | C | C | C | G | G | T | G | C | A | G | T | A | T | G | A |
| | A | A | G | A | A | G | A | T | T | G | A | A | A | T | C | A | A | A | T | C | 1463 wt | T | A | A | T | G | C | C | C | G | G | T | G | C | A | G | T | A | T | G | A |
| | A | A | G | A | A | G | A | T | T | G | A | A | A | T | C | A | A | A | T | C | | T | A | A | C | G | C | C | C | G | G | T | G | C | A | G | T | A | T | G | A |
| | A | A | G | T | A | G | A | T | T | G | A | A | A | T | C | A | A | A | T | C | | T | T | C | C | G | C | C | C | G | G | T | G | C | A | G | T | A | T | G | A |
| | A | A | G | A | A | G | A | T | T | G | A | A | A | T | C | A | A | A | T | C | | T | T | A | C | G | C | C | C | G | G | T | G | C | A | G | T | A | T | G | A |
| | | | | | | | | | | | | | | | | | | | | | | T | T | A | C | G | C | C | C | G | G | T | G | C | A | G | T | A | T | G | A |
| dcuB123-142 wt | C | C | T | T | C | A | G | C | C | A | G | G | T | A | A | A | C | C | A | C | | T | T | A | C | G | C | C | C | G | G | T | G | C | A | G | T | A | T | G | A |
| | C | A | T | T | C | A | G | A | C | A | G | G | T | A | A | A | C | C | A | C | | T | T | C | A | G | C | C | C | G | G | T | G | C | A | G | T | A | T | G | A |
| | C | A | T | T | A | A | G | C | C | A | G | G | T | A | A | A | C | C | A | C | | T | A | C | T | G | C | C | C | G | G | T | G | C | A | G | T | A | T | G | A |
| | C | T | T | T | C | A | G | C | C | A | G | G | T | A | A | A | C | C | A | C | | T | T | T | C | G | C | C | C | G | G | T | G | C | A | G | T | A | T | G | A |
| | T | A | T | T | C | A | G | C | C | A | G | G | T | A | A | A | C | C | A | C | | T | T | T | C | G | C | C | C | G | G | T | G | C | A | G | T | A | T | G | A |
| | C | T | T | T | C | A | G | C | C | A | G | G | T | A | A | A | C | C | A | C | | T | A | C | C | G | C | C | C | G | G | T | G | C | A | G | T | A | T | G | A |
| | C | A | T | T | C | A | G | C | C | A | G | G | T | A | A | A | C | C | A | C | | T | A | C | C | G | C | C | C | G | G | T | G | C | A | G | T | A | T | G | A |
| | C | T | T | T | C | A | G | C | C | A | G | G | T | A | A | A | C | C | A | C | | T | A | C | C | G | C | C | C | G | G | T | G | C | A | G | T | A | T | G | A |
| | C | T | T | T | C | A | G | C | C | A | G | G | T | A | A | A | C | C | A | C | | T | A | C | C | G | C | C | C | G | G | T | G | C | A | G | T | A | T | G | A |
| | | | | | | | | | | | | | | | | | | | | | | T | T | T | T | G | C | C | C | G | G | T | G | C | A | G | T | A | T | G | A |
| dcuC970-989 wt | G | C | T | C | A | G | G | G | G | C | T | T | A | G | C | A | C | C | A | T | | T | T | T | C | G | C | C | C | G | G | T | G | C | A | G | T | A | T | G | A |
| | G | A | T | C | A | G | G | G | G | C | T | T | A | G | C | A | C | C | A | T | | T | T | A | C | G | C | C | C | G | G | T | G | C | A | G | T | A | T | G | A |
| | G | A | T | C | A | G | G | G | G | C | T | T | A | G | C | A | C | C | A | T | | T | T | T | C | G | C | C | C | G | G | T | G | C | A | G | T | A | T | G | A |
| | G | C | T | A | A | G | G | G | G | C | T | T | A | G | C | A | C | C | A | T | | T | T | A | C | G | C | C | C | G | G | T | G | C | A | G | T | A | T | G | A |
| | G | A | T | C | A | G | G | G | G | C | T | T | A | G | C | A | C | C | A | T | | T | T | A | C | G | C | C | C | G | G | T | G | C | A | G | T | A | T | G | A |
| | G | T | T | C | A | G | G | G | G | C | T | T | A | G | C | A | C | C | A | T | | T | A | A | C | G | C | C | C | G | G | T | G | C | A | G | T | A | T | G | A |
| | | | | | | | | | | | | | | | | | | | | | | T | T | A | C | G | C | C | C | G | G | T | G | C | A | G | T | A | T | G | A |
| dcuD801-820 wt | G | C | A | G | T | C | A | G | A | A | C | T | G | C | A | T | C | T | G | G | | T | T | A | C | G | C | C | C | G | G | T | G | C | A | G | T | A | T | G | A |
| | G | A | A | G | T | C | A | G | A | A | C | T | G | C | A | T | C | T | G | G | | T | A | T | C | G | C | C | C | G | G | T | G | C | A | G | T | A | T | G | A |
| | G | A | A | G | T | T | A | G | A | A | C | T | G | C | A | T | C | T | G | G | | T | T | A | C | G | C | C | C | G | G | T | G | C | A | G | T | A | T | G | A |
| | G | A | A | G | T | C | A | G | A | A | C | T | G | C | A | T | C | T | G | G | | T | T | A | C | G | C | C | C | G | G | T | G | C | A | G | T | A | T | G | A |
| | | | | | | | | | | | | | | | | | | | | | | T | T | T | C | G | C | C | C | G | G | T | G | C | A | G | T | A | T | G | A |
| relA 129-148 wt | G | C | A | A | C | A | G | A | C | G | C | A | G | G | G | G | C | A | T | C | | T | T | T | C | G | C | C | C | G | G | T | G | C | A | G | T | A | T | G | A |
| | G | A | A | A | C | A | G | A | C | G | C | A | G | G | G | G | C | A | T | C | | T | T | C | A | G | C | C | C | G | G | T | G | C | A | G | T | A | T | G | A |
| | G | A | A | A | C | A | G | A | C | G | C | A | G | G | G | G | C | A | T | C | | T | C | T | C | G | C | C | C | G | G | T | G | C | A | G | T | A | T | G | A |
| | G | T | A | A | C | A | G | A | C | G | C | A | G | G | G | G | C | A | T | C | | T | A | A | C | G | C | C | C | G | G | T | G | C | A | G | T | A | T | G | A |
| | G | T | A | A | C | A | G | A | C | G | C | A | G | G | G | G | C | A | T | C | | T | T | T | C | G | C | C | C | G | G | T | G | C | A | G | T | A | T | G | A |
| | | | | | | | | | | | | | | | | | | | | | | T | A | C | T | G | C | C | C | G | G | T | G | C | A | G | T | A | T | G | A |
| rpoS175-194 wt | C | A | G | C | T | T | T | A | C | C | T | T | G | G | T | G | A | G | A | T | | T | A | C | A | G | C | C | C | G | G | T | G | C | A | G | T | A | T | G | A |
| | C | A | G | A | T | T | T | A | C | C | T | T | G | G | T | G | A | G | A | T | | T | C | A | C | G | C | C | C | G | G | T | G | C | A | G | T | A | T | G | A |
| | | | | | | | | | | | | | | | | | | | | | | T | T | A | C | G | C | C | C | G | G | T | G | C | A | G | T | A | T | G | A |
| E.coli MG1655 | | | | | | | | | | | | | | | | | | | | | | T | T | T | C | G | C | C | C | G | G | T | G | C | A | G | T | A | T | G | A |
| dcuA643-662 wt | A | A | G | C | A | G | A | T | T | G | A | A | A | T | C | A | A | A | T | C | | T | T | A | C | G | C | C | C | G | G | T | G | C | A | G | T | A | T | G | A |
| | A | A | G | A | A | G | A | T | T | G | A | A | A | T | C | A | A | A | T | C | | T | A | A | C | G | C | C | C | G | G | T | G | C | A | G | T | A | T | G | A |
| | A | A | G | A | A | G | A | T | T | G | A | A | A | T | C | A | A | A | T | C | | T | A | C | C | G | C | C | C | G | G | T | G | C | A | G | T | A | T | G | A |
| | A | A | G | A | A | G | A | T | T | G | A | A | A | T | C | A | A | A | T | C | | T | A | A | C | G | C | C | C | G | G | T | G | C | A | G | T | A | T | G | A |
| | | | | | | | | | | | | | | | | | | | | | | T | A | C | A | G | C | C | C | G | G | T | G | C | A | G | T | A | T | G | A |
| dcuC970-989 wt | G | C | T | C | A | G | G | G | G | C | T | T | A | G | C | A | C | C | A | T | | T | A | C | C | G | C | C | C | G | G | T | G | C | A | G | T | A | T | G | A |
| | G | A | T | C | A | G | G | G | G | C | T | T | A | G | C | A | C | C | A | T | | | | | | | | | | | | | | | | | | | | | |
| | G | A | T | C | A | G | G | G | G | C | T | T | A | G | C | A | C | C | A | T | lacZ 2293- | T | T | T | C | T | T | T | C | A | C | A | G | A | T | G | T | G | G | A | T |
| | G | A | T | C | A | G | G | G | G | C | T | T | A | G | C | A | C | C | A | T | 2312 wt | T | T | T | A | T | T | T | C | A | C | A | G | A | T | G | T | G | G | A | T |
| | G | A | T | C | A | G | G | G | G | C | T | T | A | G | C | A | C | C | A | T | | T | T | T | A | T | T | T | C | A | C | A | G | A | T | G | T | G | G | A | T |
| | G | T | T | C | A | G | G | G | G | C | T | T | A | G | C | A | C | C | A | T | | T | T | T | T | T | T | T | C | A | C | A | G | A | T | G | T | G | G | A | T |
| | G | A | T | C | A | G | G | G | G | C | T | T | A | G | C | A | C | C | A | T | | T | T | T | A | T | T | T | C | A | C | A | G | A | T | G | T | G | G | A | T |
| | G | A | T | C | A | G | G | G | G | C | T | T | A | G | C | A | C | C | A | T | | T | T | T | T | T | T | T | C | A | C | A | G | A | T | G | T | G | G | A | T |
| | | | | | | | | | | | | | | | | | | | | | | T | T | T | A | T | T | T | C | A | C | A | G | A | T | G | T | G | G | A | T |
| lacZ 1431-1450 wt | A | T | C | T | G | T | C | G | A | T | C | C | T | T | C | C | C | G | C | C | | T | T | T | A | T | T | T | C | A | C | A | G | A | T | G | T | G | G | A | T |
| | A | T | T | T | G | T | C | G | A | T | C | C | T | T | C | C | C | G | C | C | | T | T | T | T | T | T | T | C | A | C | A | G | A | T | G | T | G | G | A | T |
| | A | T | A | T | G | T | A | G | A | T | C | C | T | T | C | C | C | G | C | C | | T | T | T | A | T | T | T | C | A | C | A | G | A | T | G | T | G | G | A | T |
| | A | T | A | T | G | T | A | G | A | T | C | C | T | T | C | C | C | G | C | C | | | | | | | | | | | | | | | | | | | | | |
| | A | T | A | T | G | T | C | G | A | T | C | C | T | T | C | C | C | G | C | C | lacZ 1640- | T | T | G | G | C | G | G | T | T | T | C | G | C | T | A | A | A | T | A | C |
| | A | T | A | T | G | T | C | G | A | T | C | C | T | T | C | C | C | G | C | C | 1659 wt | T | T | G | G | A | G | G | T | T | T | C | G | C | T | A | A | A | T | A | C |
| | A | T | T | T | G | T | C | G | A | T | C | C | T | T | C | C | C | G | C | C | | T | T | G | G | T | G | G | T | T | T | C | G | C | T | A | A | A | T | A | C |
| | A | T | A | T | G | T | T | G | A | T | C | C | T | T | C | C | C | G | C | C | | | | | | | | | | | | | | | | | | | | | |
| | | | | | | | | | | | | | | | | | | | | | lacZ 1608- | T | T | G | C | G | A | A | T | A | C | G | C | C | C | A | C | G | C | G | A |
| | | | | | | | | | | | | | | | | | | | | | 1627 wt | T | T | G | A | G | A | A | T | A | C | G | C | C | C | A | C | G | C | G | A |
| | | | | | | | | | | | | | | | | | | | | | | T | T | G | A | G | A | A | T | A | C | G | C | C | C | A | C | G | C | G | A |
| | | | | | | | | | | | | | | | | | | | | | | T | T | G | A | G | A | A | T | A | C | G | C | C | C | A | C | G | C | G | A |
| | | | | | | | | | | | | | | | | | | | | | | T | T | G | A | G | A | A | T | A | C | G | C | C | C | A | C | G | C | G | A |
| | | | | | | | | | | | | | | | | | | | | | | T | T | G | T | G | A | A | T | A | C | G | C | C | C | A | C | G | C | G | A |
| | | | | | | | | | | | | | | | | | | | | | | T | T | G | A | G | A | A | T | A | A | G | C | C | C | A | C | G | C | G | A |
| | | | | | | | | | | | | | | | | | | | | | | T | T | G | T | G | A | A | T | A | C | G | C | C | C | A | C | G | C | G | A |

The left part of Table 2 shows the SEQ ID NO: 30-80 respectively; the right part shows the SEQ ID NO: 81-147 respectively.

### II. Test Results

In the wild Escherichia coli MG1655 and ATCC 8739, the cytosine deaminase (AID) and nCas9 fusion expression system is used for the site-directed base editing by selecting different genes such as dcuA, dcuB, dcuC, dcuD, relA, rpoS and lacZ and different sites of the same gene, respectively.

The editing results are as shown in Table 2. The results show that among the 7 target sites of the Escherichia coli MG1655, a total of 51 bases C were mutated to bases T and 70 bases C were mutated to bases A. Among the 6 target sites of the Escherichia coli ATCC 8739, a total of 10 bases C were mutated to bases T and 19 bases C were mutated to bases A. Wherein the C-to-T mutations accounted for 40.7% (61/150) of total mutations, and C-to-A mutations accounted for 59.3% (89/150) of the total mutations. The above results indicate that the base editing system composed of cytosine deaminase (AID) and nCas9 can not only realize the base substitution of cytosine (C) to thymine (T), but also realize the base substitution of cytosine (C) to adenine (A).

### EXAMPLE 2

### A Method for Improving a Base Editing Efficiency of Mutating a Target Bace C to A in Escherichia coli

In order to improve a base editing efficiency of mutating cytosine (C) to adenine (A) at a specific site in Escherichia coli, the fusion expression of cytosine deaminase (AID), nCas9 and uracil DNA glycosidase was performed in Escherichia coli, so that the base editing efficiency of mutating cytosine (C) to adenine (A) can reach 94.5%.

### I. Test Methods

The pUNG_nCas9_AID plasmid containing a cytosine deaminase (AID), nCas9 and uracil DNA glycosidase fusion expression system and the Escherichia coli gRNA plasmid containing different target sites were introduced into wild Escherichia coli MG1655, plating was performed after culturing for 24 h, and a part of colonies were randomly selected for PCR detection and sequencing.

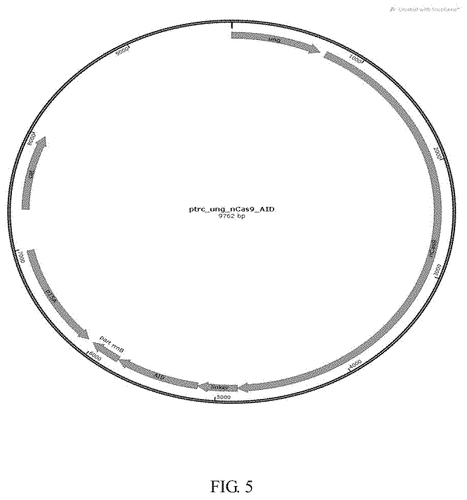

A map of the pUNG_nCas9_AID plasmid was shown in FIG. 5, and its nucleotide sequence was as shown in SEQ ID NO: 3; wherein sites 1-687 represented an encoding gene sequence of the uracil DNA glycosidase, sites 736-4,839 represented an encoding gene sequence of nCas9, sites 5,140-5,781 represented an encoding gene sequence of the cytosine deaminase (AID), sites 7,344-8,003 were chloramphenicol genes, and sites 6,070-6,980 were origins of replication. The pUNG_nCas9_AID plasmid expressed a fusion protein composed of cytosine deaminase (AID), nCas9 and uracil DNA glycosidase.

The Escherichia coli gRNA plasmid containing different target sites was the gRNA plasmid targeting at different sites of the lacZ gene. The specific target sites of the gRNA plasmid were as shown in Table 3.

TABLE 3
Specific Target Sites of the gRNA Plasmid

| | N | N | N | N | N | N | N | N | N | N | N | N | N | N | N | N | N | N | N | N | E.coli | N | N | N | N | N | N | N | N | N | N | N | | | | | | | | N | |
|---|---|---|---|---|---|---|---|---|---|---|---|---|---|---|---|---|---|---|---|---|---|---|---|---|---|---|---|---|---|---|---|---|---|---|---|---|---|---|---|---|---|
| E.coli MG1655 | 20 | 19 | 18 | 17 | 16 | 15 | 14 | 13 | 12 | 11 | 10 | 9 | 8 | 7 | 6 | 5 | 4 | 3 | 2 | 1 | MG1655 | 20 | 19 | 18 | 17 | 16 | 15 | 14 | 13 | 12 | 11 | 10 | N9 | N8 | N7 | N6 | N5 | N4 | N3 | 2 | N1 |
| lacZ 1444- | T | C | C | C | G | C | C | C | G | G | T | G | C | A | G | T | A | T | G | A | lacZ 1431- | A | T | C | T | G | T | C | G | A | T | C | C | T | T | C | C | C | G | C | C |
| 1463 wt | T | C | C | C | G | C | C | C | G | G | T | G | C | A | G | T | A | T | G | A | 1450 wt | A | T | C | T | G | T | C | G | A | T | C | C | T | T | C | C | C | G | C | C |
| | T | C | C | C | G | C | C | C | G | G | T | G | C | A | G | T | A | T | G | A | | A | T | C | T | G | T | C | G | A | T | C | C | T | T | C | C | C | G | C | C |
| | T | C | C | C | G | C | C | C | G | G | T | G | C | A | G | T | A | T | G | A | | A | T | C | T | G | T | C | G | A | T | C | C | T | T | C | C | C | G | C | C |
| | T | C | C | C | G | C | C | C | G | G | T | G | C | A | G | T | A | T | G | A | | A | T | C | T | G | T | C | G | A | T | C | C | T | T | C | C | C | G | C | C |
| | T | C | C | C | G | C | C | C | G | G | T | G | C | A | G | T | A | T | G | A | | A | T | C | T | G | T | C | G | A | T | C | C | T | T | C | C | C | G | C | C |
| | T | C | C | C | G | C | C | C | G | G | T | G | C | A | G | T | A | T | G | A | | A | T | C | T | G | T | C | G | A | T | C | C | T | T | C | C | C | G | C | C |
| | T | C | C | C | G | C | C | C | G | G | T | G | C | A | G | T | A | T | G | A | | A | T | C | T | G | T | C | G | A | T | C | C | T | T | C | C | C | G | C | C |
| | T | C | C | C | G | C | C | C | G | G | T | G | C | A | G | T | A | T | G | A | | A | T | C | T | G | T | C | G | A | T | C | C | T | T | C | C | C | G | C | C |
| | T | C | C | C | G | C | C | C | G | G | T | G | C | A | G | T | A | T | G | A | | A | T | C | T | G | T | C | G | A | T | C | C | T | T | C | C | C | G | C | C |
| | T | C | C | C | G | C | C | C | G | G | T | G | C | A | G | T | A | T | G | A | | A | T | C | T | G | T | C | G | A | T | C | C | T | T | C | C | C | G | C | C |
| | T | C | C | C | G | C | C | C | G | G | T | G | C | A | G | T | A | T | G | A | | A | T | C | T | G | T | C | G | A | T | C | C | T | T | C | C | C | G | C | C |
| | T | C | C | C | G | C | C | C | G | G | T | G | C | A | G | T | A | T | G | A | | A | T | C | T | G | T | C | G | A | T | C | C | T | T | C | C | C | G | C | C |
| | T | C | C | C | G | C | C | C | G | G | T | G | C | A | G | T | A | T | G | A | | A | T | C | T | G | T | C | G | A | T | C | C | T | T | C | C | C | G | C | C |
| | T | C | C | C | G | C | C | C | G | G | T | G | C | A | G | T | A | T | G | A | | | | | | | | | | | | | | | | | | | | | |
| | T | C | C | C | G | C | C | C | G | G | T | G | C | A | G | T | A | T | G | A | lacZ 1431- | T | T | G | C | G | A | A | T | A | C | G | C | C | C | A | C | G | C | G | A |
| | T | C | C | C | G | C | C | C | G | G | T | G | C | A | G | T | A | T | G | A | 1450 wt | T | T | G | A | G | A | A | T | A | C | G | C | C | C | A | C | G | C | G | A |
| | T | C | C | C | G | C | C | C | G | G | T | G | C | A | G | T | A | T | G | A | | T | T | G | A | G | A | A | T | A | C | G | C | C | C | A | C | G | C | G | A |
| | T | C | C | C | G | C | C | C | G | G | T | G | C | A | G | T | A | T | G | A | | T | T | G | A | G | A | A | T | A | C | G | C | C | C | A | C | G | C | G | A |
| | T | C | C | C | G | C | C | C | G | G | T | G | C | A | G | T | A | T | G | A | | T | T | G | A | G | A | A | T | A | C | G | C | C | C | A | C | G | C | G | A |
| | T | C | C | C | G | C | C | C | G | G | T | G | C | A | G | T | A | T | G | A | | T | T | G | A | G | A | A | T | A | C | G | C | C | C | A | C | G | C | G | A |
| | T | C | C | C | G | C | C | C | G | G | T | G | C | A | G | T | A | T | G | A | | T | T | G | A | G | A | A | T | A | C | G | C | C | C | A | C | G | C | G | A |
| | T | C | C | C | G | C | C | C | G | G | T | G | C | A | G | T | A | T | G | A | | T | T | G | A | G | A | A | T | A | C | G | C | C | C | A | C | G | C | G | A |
| | T | C | C | C | G | C | C | C | G | G | T | G | C | A | G | T | A | T | G | A | | T | T | G | A | G | A | A | T | A | C | G | C | C | C | A | C | G | C | G | A |
| | T | C | C | C | G | C | C | C | G | G | T | G | C | A | G | T | A | T | G | A | | T | T | G | A | G | A | A | T | A | C | G | C | C | C | A | C | G | C | G | A |
| | T | C | C | C | G | C | C | C | G | G | T | G | C | A | G | T | A | T | G | A | | T | T | G | A | G | A | A | T | A | C | G | C | C | C | A | C | G | C | G | A |
| | T | C | C | C | G | C | C | C | G | G | T | G | C | A | G | T | A | T | G | A | | T | T | G | A | G | A | A | T | A | C | G | C | C | C | A | C | G | C | G | A |
| | T | C | C | C | G | C | C | C | G | G | T | G | C | A | G | T | A | T | G | A | | T | T | G | A | G | A | A | T | A | C | G | C | C | C | A | C | G | C | G | A |
| | T | C | C | C | G | C | C | C | G | G | T | G | C | A | G | T | A | T | G | A | | T | T | G | A | G | A | A | T | A | C | G | C | C | C | A | C | G | C | G | A |
| | T | C | C | C | G | C | C | C | G | G | T | G | C | A | G | T | A | T | G | A | | T | T | G | A | G | A | A | T | A | C | G | C | C | C | A | C | G | C | G | A |
| | T | C | C | C | G | C | C | C | G | G | T | G | C | A | G | T | A | T | G | A | | T | T | G | A | G | A | A | T | A | C | G | C | C | C | A | C | G | C | G | A |
| | T | C | C | C | G | C | C | C | G | G | T | G | C | A | G | T | A | T | G | A | | T | T | G | A | G | A | A | T | A | C | G | C | C | C | A | C | G | C | G | A |
| | T | C | C | C | G | C | C | C | G | G | T | G | C | A | G | T | A | T | G | A | | T | T | G | A | G | A | A | T | A | C | G | C | C | C | A | C | G | C | G | A |
| | T | C | C | C | G | C | C | C | G | G | T | G | C | A | G | T | A | T | G | A | | | | | | | | | | | | | | | | | | | | | |
| | T | C | C | C | G | C | C | C | G | G | T | G | C | A | G | T | A | T | G | A | lacz 984- | C | T | G | C | G | A | T | G | T | C | G | G | T | T | T | C | C | G | C | G |
| | T | C | C | C | G | C | C | C | G | G | T | G | C | A | G | T | A | T | G | A | 1003 wt | C | T | G | A | G | A | T | G | T | C | G | G | T | T | T | C | C | G | C | G |
| | T | C | C | C | G | C | C | C | G | G | T | G | C | A | G | T | A | T | G | A | | C | T | G | A | G | A | T | G | T | C | G | G | T | T | T | C | C | G | C | G |
| | T | C | C | C | G | C | C | C | G | G | T | G | C | A | G | T | A | T | G | A | | C | T | G | A | G | A | T | G | T | C | G | G | T | T | T | C | C | G | C | G |
| | T | C | C | C | G | C | C | C | G | G | T | G | C | A | G | T | A | T | G | A | | C | T | G | A | G | A | T | G | T | C | G | G | T | T | T | C | C | G | C | G |
| | T | C | C | C | G | C | C | C | G | G | T | G | C | A | G | T | A | T | G | A | | C | T | G | A | G | A | T | G | T | C | G | G | T | T | T | C | C | G | C | G |
| | | | | | | | | | | | | | | | | | | | | | | C | T | G | A | G | A | T | G | T | C | G | G | T | T | T | C | C | G | C | G |
| | | | | | | | | | | | | | | | | | | | | | | C | T | G | A | G | A | T | G | T | C | G | G | T | T | T | C | C | G | C | G |
| | | | | | | | | | | | | | | | | | | | | | | C | T | G | A | G | A | T | G | T | C | G | G | T | T | T | C | C | G | C | G |
| | | | | | | | | | | | | | | | | | | | | | | C | T | G | A | G | A | T | G | T | C | G | G | T | T | T | C | C | G | C | G |
| | | | | | | | | | | | | | | | | | | | | | | C | T | G | A | G | A | T | G | T | C | G | G | T | T | T | C | C | G | C | G |
| | | | | | | | | | | | | | | | | | | | | | | C | T | G | A | G | A | T | G | T | C | G | G | T | T | T | C | C | G | C | G |
| | | | | | | | | | | | | | | | | | | | | | | C | T | G | A | G | A | T | G | T | C | G | G | T | T | T | C | C | G | C | G |
| | | | | | | | | | | | | | | | | | | | | | | C | T | G | A | G | A | T | G | T | C | G | G | T | T | T | C | C | G | C | G |
| | | | | | | | | | | | | | | | | | | | | | | C | T | G | A | G | A | T | G | T | C | G | G | T | T | T | C | C | G | C | G |

The left part of table 3 shows the SEQ ID NO: 148-187 respectively; the right part shows the SEQ ID NO: 188-234 respectively.

### II. Test Results

In the wild Escherichia coli MG1655, 4 sites of the lacZ gene were selected for site-directed base editing, and the base editing efficiency was calculated [base editing efficiency=(number of positive strains with target base substitutions/total number of positive strains analyzed)×100%].

The editing results are as shown in Table 7. The results show that among the 4 target sites of the Escherichia coli MG1655, a total of 121 bases C were mutated to bases A, 5 bases C were mutated to bases T, and 2 bases C were mutated to bases G. The C-to-A mutations accounted for 94.5% (121/128) of total mutations. The above results indicate that the base editing system composed of cytosine deaminase (AID), nCas9 and uracil DNA glycosidase can significantly improve the base editing efficiency of mutating C to A.

### EXAMPLE 3

### A Method for Improving a Base Editing Efficiency of Mutating a Target Base C to G in Mammalian Cells

In a literature (Komor, A. C., Kim, Y. B., Packer, M. S., Zuris, J. A. & Liu, D. R. Programmable editing of a target base in genomic DNA without double-stranded DNA cleavage. Nature 533, 420-424 (2016)), it has been found that the fusion expression of cytosine deaminase (APOBEC) and nCas9 in mammalian cells can achieve site-directed substitutions of cytosine (C) to thymine (T) as well as cytosine (C) to guanine (G) at a specific site of the mammalian cells; wherein the C-to-T mutations accounted for 89.6% of total mutations and C-to-G mutations accounted for 10.4% of the total mutations.

In order to improve a base editing efficiency of mutating cytosine (C) to guanine (G) at a specific site in mammalian cells, the fusion expression of cytosine deaminase (APOBEC), nCas9 and uracil DNA glycosidase was performed in mammalian cells, so that the base editing efficiency of mutating cytosine (C) to guanine (G) can reach 95.2%.

### I. Test Methods

The pAPOBEC_nCas9_UGI plasmid containing a cytosine deaminase (APOBEC), nCas9 and uracil DNA glycosylase inhibitory protein (UGI) fusion expression system and the pAPOBEC_nCas9_UNG palsmid containing a cytosine deaminase (APOBEC), nCas9 and uracil DNA glycosidase fusion expression system were respectively transfected with mammalian cells gRNA plasmid containing targeting sites into HEK293T or Hela cells using a Lipofectamine 2000 (Life, Invitrogen, 11668019) reagent, and cells genome DNA was extracted after transfection for 96 h for PCR detection and sequencing of an edited site. Two parallel tests (test 1 and test 2) were performed for each type of cells using each combination method.

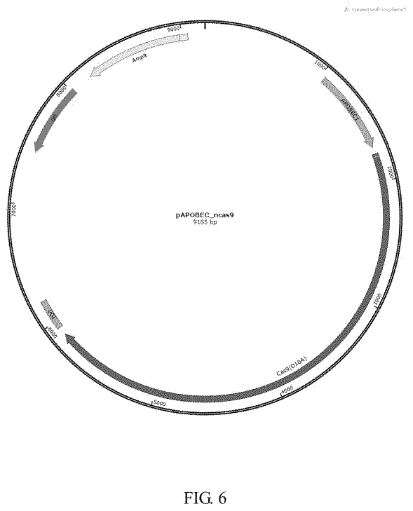

A map of the pAPOBEC_nCas9_UGI plasmid was as shown in FIG. 6, and its nucleotide sequence was as shown in SEQ ID NO: 4; wherein sites 1,038-1,721 represented an encoding gene sequence of the cytosine deaminase (APOBEC1), sites 1,773-5,873 represented an encoding gene sequence of the nCas9, sites 5,943-6,191 represented an encoding gene sequence of the uracil DNA glycosylase inhibitory protein (UGI), sites 7,430-8,018 were replicons for the amplification of Escherichia coli, and sites 8,189-9,049 were ampicillin resistance genes for the amplification of Escherichia coli. The pAPOBEC_nCas9_UGI plasmid expressed a fusion protein composed of cytosine deaminase (APOBEC), nCas9 and uracil DNA glycosylase inhibitory protein (UGI).

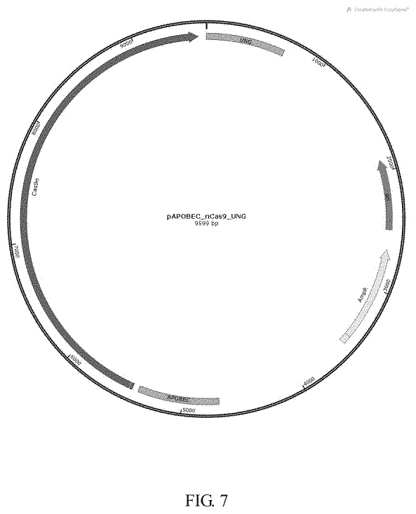

A map of the pAPOBEC_nCas9_UNG plasmid was as shown in FIG. 7, and its nucleotide sequence was as shown in SEQ ID NO: 5; wherein sites 1-663 represented an encoding gene sequence of the uracil DNA glycosidase, sites 1,902-2,490 were replicons for the amplification of Escherichia coli, sites 2,661-3,521 were ampicillin resistance genes for the amplification of Escherichia coli, sites 4,695-5,375 represented an encoding gene sequence of the cytosine deaminase (APOBEC), and sites 5,430-9,530 represented an encoding gene sequence of nCas9. The pAPOBEC_nCas9_UNG plasmid expressed a fusion protein composed of cytosine deaminase (APOBEC), nCas9 and uracil DNA glycosidase.

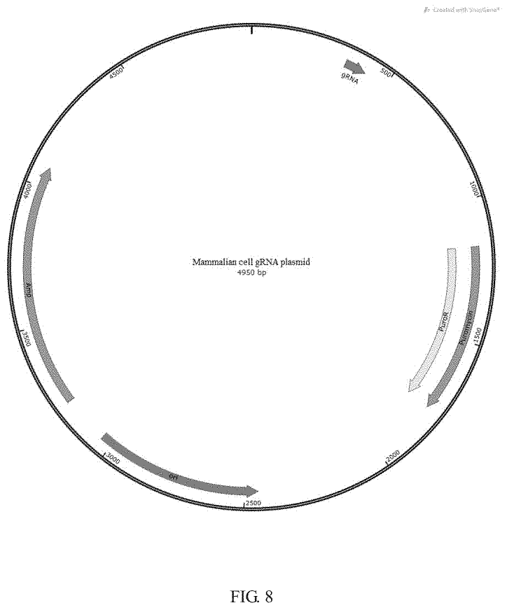

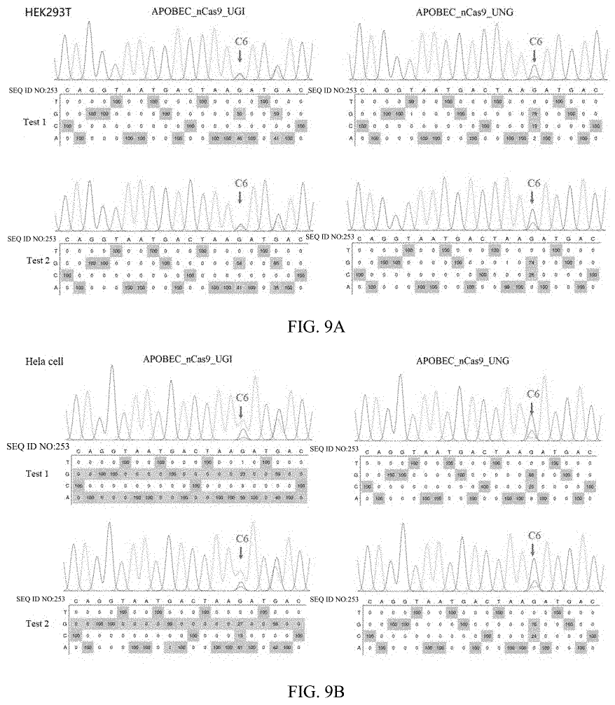

A map of the mammalian cells gRNA plasmid containing targeting sites (targeting sites 42,220-42,239 of RNF2 gene) was as shown in FIG. 8, and its nucleotide sequence was as shown in SEQ ID NO: 6; wherein sites 322-341 represented the target sequence, sites 342-417 represented a gRNA sequence, sites 1,167-1,766 are purinomycin genes of the mammalian cells, sites 2,453-3,041 were replicons for the amplification of Escherichia coli, and sites 3,212-4,072 were ampicillin genes for the amplification of Escherichia coli. In this example or hereinafter, the mammalian cells gRNA plasmids targeting at other sites can be obtained simply by substituting the target sequence in the gRNA plasmid shown in SEQ ID NO: 6 by the target sequence of other genes or other target sequences of the same gene.

### II. Test Results

In the mammalian cells HEK293T or Hela cells, the RNF2 gene sites were selected for site-directed base editing, PCR was performed on the target sites, and deep sequencing analysis was performed on the PCR products, with more than 100,000 reads for the deep sequencing of each PCR product; the base editing efficiency was calculated according to the following formula: base editing efficiency=(number of reads with target base substitutions/total number of reads analyzed))×100%. The sequencing primer sequences were as follows:

RNF2-deep-F1:
(SEQ ID NO: 235)
CGTGTATCACCACGCC;
RNF2-deep-R1:
(SEQ ID NO: 236)
CAATACAAAGATTTTCCTAC;
RNF2-deep-F2:
(SEQ ID NO: 237)
TGAGATGGAGTCTTGCTGTG;
RNF2-deep-R2:
(SEQ ID NO: 238)
CAGGCAGATCACAAGGTCAG.

The editing results are as shown in Table 9. The results show that the base editing efficiency of mutating a base C to G at the C6 site in the HEK293T cells is increased from 10.4% to 95.2%, and the base editing efficiency of mutating C to G at C6 site in the Hela cells increased from 14.8% to 87.9%.

### EXAMPLE 4

### A Base Editing Method of Mutating Any Base to Any Base in Escherichia coli

A combination of a base editing system for mutating C to A, a base editing system for mutating C to T and a base editing system for mutating A to G realizes the mutation of a base A, T, C or G to any base in Escherichia coli, as shown in FIG. 1.

### I. Test Methods

### 1. Mutation from a Base C to Any Base

The pUNG_nCas9_AID plasmid containing a cytosine deaminase (AID), nCas9 and uracil DNA glycosidase fusion expression system and an Escherichia coli gRNA plasmid (the target sequence in the gRNA plasmid was TTT [symbol] TTTCACAGATGTGGAT (SEQ ID NO:239), in which underlined bases were specific sites to be edited) were introduced into wild Escherichia coli MG1655, plating was performed after culturing for 24 h, and a part of colonies were randomly selected for PCR detection and sequencing of the edited sites, and the strains with C mutated to A at the specific sites were screened out respectively so as to realize the editing from a base C to a base A.

The pnCas9_AID plasmid containing a cytosine deaminase (AID) and nCas9 fusion expression system and the Escherichia coli gRNA plasmid (the target sequence in the gRNA plasmid was TTT [symbol] TTTCACAGATGTGGAT (SEQ ID NO:239), in which underlined bases were specific sites to be edited) were introduced into wild Escherichia coli MG1655, plating was performed after culturing for 24 h, and a part of colonies were randomly selected for PCR detection and sequencing, and the strains with C mutated to T at the specific sites were screened out respectively so as to realize the editing from a base C to a base T.

The screened strains with C mutated to A were cultured, and the plasmid was discarded; then the pTadA_nCas9 plasmid containing an adenine deaminase (TadA) and nCas9 fusion expression system and an Escherichia coli gRNA plasmid (the target sequence in the gRNA plasmid was TTT [symbol] TTTCACAGATGTGGAT (SEQ ID NO:240), in which underlined bases were specific sites to be edited) were introduced into the strains with C mutated to A, plating was performed after culturing for 24 h, and a part of colonies were randomly selected for PCR detection and sequencing of the edited sites, and the strains with C mutated to G at the specific sites were screened out so as to realize the editing from a base C to a base G.

### 2. Mutation from a Base T to Any Base

The pTadA_nCas9 plasmid containing an adenine deaminase (AID) and nCas9 fusion expression system and the Escherichia coli gRNA plasmid (the target sequence in the gRNA plasmid was AGGCC [symbol] ATCCGCGCCGGATG (SEQ ID NO:241), in which underlined bases were specific sites to be edited) were introduced into wild Escherichia coli MG1655, plating was performed after culturing for 24 h, and a part of colonies were randomly selected for PCR detection and sequencing, and the strains with A mutated to G at the specific sites were screened out so as to realize the editing from a base T to a base C.

The screened strains with C mutated to A were cultured without antibiotic, and the plasmid was discarded; then the pUNG_nCas9_AID plasmid containing a cytosine deaminase (AID), nCas9 and UNG fusion expression system and the Escherichia coli gRNA plasmid (the target sequence in the gRNA plasmid was GAT [symbol] GGCCTGAACTGCCAGC(SEQ ID NO:242), in which underlined bases were specific sites to be edited) were introduced into the strains with A mutated to G, plating was performed after culturing for 24 h, a part of colonies were randomly selected for PCR detection and sequencing of the edited sites, and the strains with C mutated to A at the specific sites were screened out so as to realize the editing from a base T to a base A.

The screened strains with C mutated to A were cultured without antibiotic, and the plasmid was discarded; then the pTadA_nCas9 plasmid containing an adenine deaminase (TadA) and nCas9 fusion expression system and the Escherichia coli gRNA plasmid (the target sequence in the gRNA plasmid was GAT [symbol] GGCCTGAACTGCCAGC (SEQ ID NO:243), in which underlined bases were specific sites to be edited) were introduced into the strains with C mutated to A, plating was performed after culturing for 24 h, and a part of colonies were randomly selected for PCR detection and sequencing of the edited sites, and the strains with A mutated to G at the specific sites were screened out so as to realize the editing from a base T to a base G.

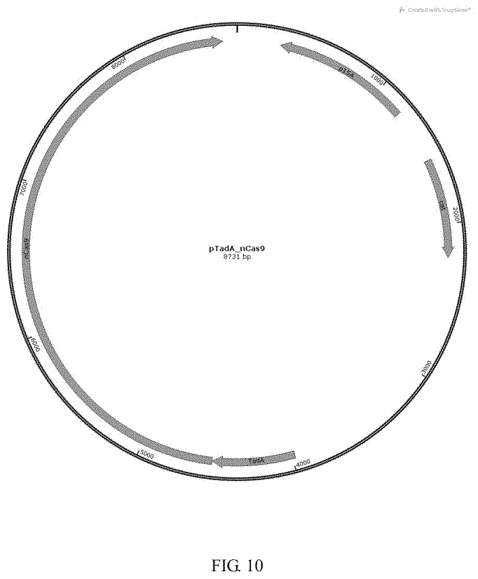

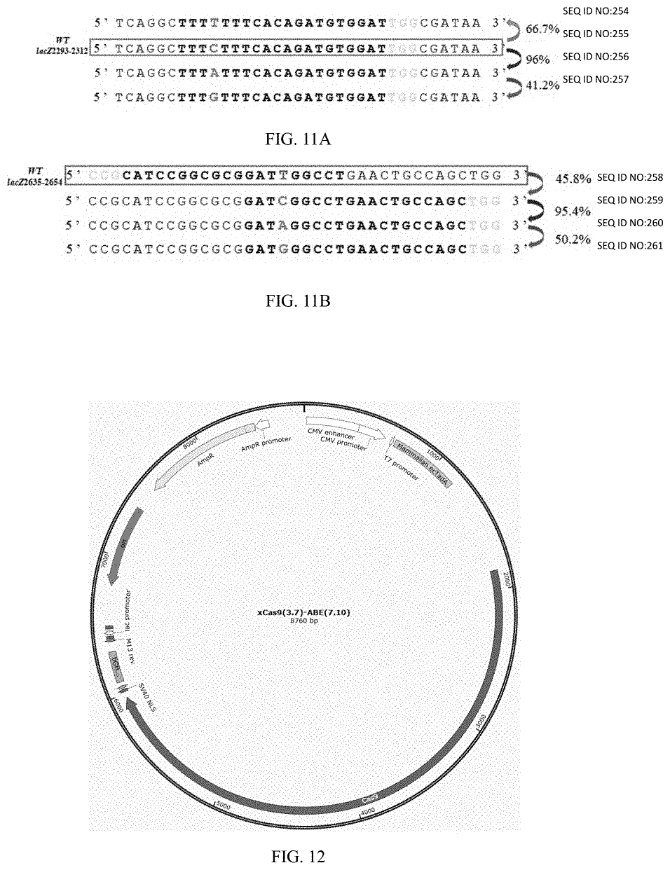

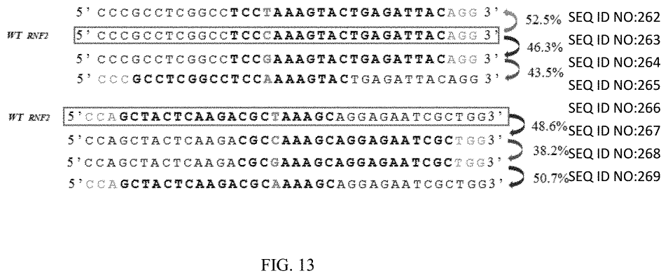

A map of the pTadA_nCas9 plasmid was as shown in FIG. 10, and its nucleotide sequence was as shown in SEQ ID NO: 7; wherein sites 3,982-4,530 represented an encoding gene sequence of the adenine deaminase (TadA), sites 4,531-8,637 represented an encoding gene sequence of the nCas9, sites 1,563-2,222 were chloramphenicol genes, and sites 289-1,199 were origins of replication. The pTadA_nCas9 plasmid expressed a fusion protein composed of adenine deaminase (TadA) and nCas9.

### II. Test Results

In the wild Escherichia coli MG1655, 2 sites of the lacZ gene were selected for editing any base, and the base editing efficiency was calculated [base editing efficiency=(number of positive strains with target base substitutions/total number of positive strains analyzed)×100%].

The editing results are as shown in Table 11. The results show that starting from the base C, the editing efficiency of mutating the base C to the base T is 66.7%, the editing efficiency of mutating the base C to the base A is 96%, and the editing efficiency of mutating the base C to the base G is 96%×41.2%=39.6%. Starting from the base T (its complementary base is the base A), the editing efficiency of mutating the base T to the base C is 45.8%, the editing efficiency of mutating the base T to the base A is 45.8%×95.4%=43.7%, and the editing efficiency of mutating the base T to the base G is 45.8%×95.4%×50.2%=21.9%.

### EXAMPLE 5

### A Base Editing Method of Mutating Any Base to Any Base in Mammalian Cells

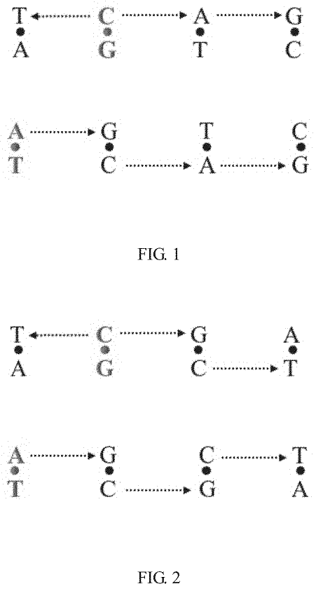

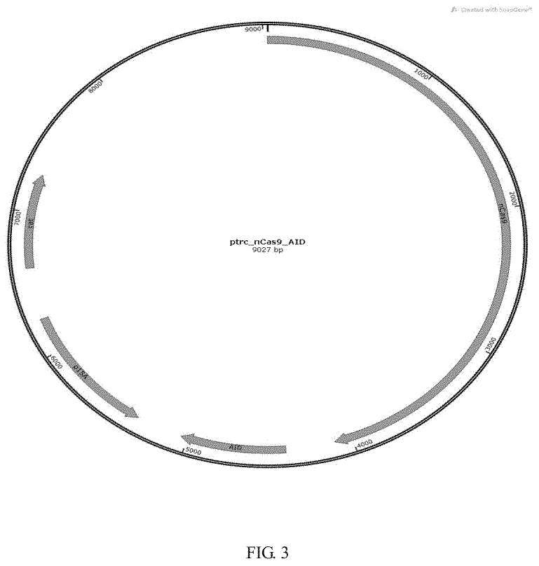

A combination of a base editing system for mutating C to G, a base editing system for mutating C to T and a base editing system for mutating A to G realizes the mutation of a base A, T, C or G to any base in mammalian cells, as shown in FIG. 2.

### I. Test methods

### 1. Mutation from a Base C to Any Base

The pAPOBEC_nCas9_UNG plasmid containing a cytosine deaminase (APOBEC), nCas9 and uracil DNA glycosidase fusion expression system and the mammalian cells gRNA plasmid (the target sequence in the gRNA plasmid was TCC [symbol] AAAGTACTGAGATTAC(SEQ ID NO:244), in which underlined bases were specific sites to be edited) were transfected with HEK293T cells, puromycin with a final concentration of 5 ug/ml was added after transfection for 24 h, and single cells were separated using a flow cytometry after 72 h and then cultured in a 96-well plate; cellular genomes were extracted after 24 h for PCR detection and sequencing, and the cells with C mutated to G at the specific sites were screened out respectively so as to realize the editing from a base C to a base G.

The pAPOBEC_nCas9_plasmid containing a cytosine deaminase (APOBEC) and nCas9 fusion expression system and the mammalian cells gRNA plasmid (the target sequence in the gRNA plasmid was TCC [symbol] AAAGTACTGAGATTAC(SEQ ID NO:244), in which underlined bases were specific sites to be edited) were transfected with HEK293T cells, puromycin with a final concentration of 5 ug/ml was added after transfection for 24 h, and single cells were separated using the flow cytometry after 72 h and then cultured in a 96-well plate; cellular genomes were extracted after 24 h for PCR detection and sequencing, and the cells with C mutated to T at the specific sites were screened out respectively so as to realize the editing from a base C to a base T.

The pAPOBEC_nCas9_UGI plasmid containing a cytosine deaminase (APOBEC), nCas9 and uracil DNA glycosylase inhibitory protein (UGI) fusion expression system and the mammalian cells gRNA plasmid (the target sequence in the gRNA plasmid was GTACTTT [symbol] GGAGGCCGAGGC(SEQ ID NO:245), in which underlined bases were specific sites to be edited) were transfected with the cells with C mutated to G, puromycin with a final concentration of 5 ug/ml was added after transfection for 24 h, and single cells were separated using the flow cytometry after 72 h and then cultured in a 96-well plate; cellular genomes were extracted after 24 h for PCR detection and sequencing, and the cells with C mutated to T at the specific sites were screened out so as to realize the editing from a base C to a base A.

### 2. Mutation from a Base T to Any Base

The xcas9 (3.7)-ABE (7.10) plasmid containing an adenine deaminase (TadA) and xCas9 (3.7) fusion expression system and the mammalian cells gRNA plasmid (the target sequence in the gRNA plasmid was GCTTT [symbol] GCGTCTTGAGTAGC(SEQ ID NO:246), in which underlined bases were specific sites to be edited) were transfected with HEK293T cells, puromycin with a final concentration of 5 ug/ml was added after transfection for 24 h, and single cells were separated using the flow cytometry after 72 h and then cultured in a 96-well plate; cellular genomes were extracted after 24 h for PCR detection and sequencing, and the cells with A mutated to G at the specific sites were screened out so as to realize the editing from a base T to a base C.

The pAPOBEC_nCas9_UNG plasmid containing a cytosine deaminase (APOBEC), nCas9 and uracil DNA glycosidase fusion expression system and the mammalian cells gRNA plasmid (the target sequence in the gRNA plasmid was CGC [symbol] AAAGCAGGAGAATCGC(SEQ ID NO:247), in which underlined bases were specific sites to be edited) were transfected with the cells with A mutated to G, puromycin with a final concentration of 5 ug/ml was added after transfection for 24 h, and single cells were separated using a flow cytometry after 72 h and then cultured in a 96-well plate; cellular genomes were extracted after 24 h for PCR detection and sequencing, and the cells with C mutated to G at the specific sites were screened out so as to realize the editing from a base T to a base G.

The pAPOBEC_nCas9 plasmid containing a cytosine deaminase (APOBEC) and nCas9 fusion expression system and the mammalian cells gRNA plasmid (the target sequence in the gRNA plasmid was GCTTT [symbol] GCGTCTTGAGTAGC(SEQ ID NO:248), in which underlined bases were specific sites to be edited) were transfected with the cells with C mutated to G, puromycin with a final concentration of 5 ug/ml was added after transfection for 24 h, and single cells were separated using the flow cytometry after 72 h and then cultured in a 96-well plate; cellular genomes were extracted after 24 h for PCR detection and sequencing, and the cells with C mutated to T at the specific sites were screened out so as to realize the editing from a base T to a base A.

A map of the xcas9 (3.7)-ABE (7.10) plasmid was as shown in FIG. 12, and its nucleotide sequence was as shown in SEQ ID NO: 7; wherein sites 676-1,176 represented an encoding gene sequence of the adenine deaminase (TadA), sites 1,867-5,967 represented an encoding gene sequence of the xCas9 (3.7), sites 7,544-8,404 were chloramphenicol genes, and sites 6,785-7,373 were origins of replication.

### II. Test Results

In the HEK293T cells, two RNF gene sites were selected for editing any base, PCR was performed on the target sites, and deep sequencing analysis was performed on the PCR products, with more than 100,000 reads for the deep sequencing of each PCR product; the base editing efficiency was calculated according to the following formula: base editing efficiency=(number of reads with target base substitutions/total number of reads analyzed)×100%. The sequencing primer sequences were as follows:

RNF2-deep-F1:
(SEQ ID NO: 249)
CGTGTATCACCACGCC;
RNF2-deep-R1:
(SEQ ID NO: 250)
CAATACAAAGATTTTCCTAC;
RNF2-deep-F2:
(SEQ ID NO: 251)
TGAGATGGAGTCTTGCTGTG;
RNF2-deep-R2:
(SEQ ID NO: 252)
CAGGCAGATCACAAGGTCAG.

The editing results are as shown in Table 13. The results show that starting from the base C, the editing efficiency of mutating the base C to the base T is 52.5%, the editing efficiency of mutating the base C to the base G is 46.3%, and the editing efficiency of mutating the base C to the base A is 46.3%×43.5%=20.1%. Starting from the base T (its complementary base is the base A), the editing efficiency of mutating the base T to the base C is 48.6%, the editing efficiency of mutating the base T to the base G is 48.6%×38.2%=18.6%, and the editing efficiency of mutating the base T to the base A is 48.6%×38.2%×50.7%=9.4%.

The foregoing descriptions are merely some of the preferred embodiments of the present invention. It should be noted that for those of ordinary skill in the art, a number of improvements and modifications can be made without departing from the technical principle of the present invention. These improvements and modifications should also fall within the protection scope of the present invention.

### INDUSTRIAL APPLICATION

The present invention provides a base editing system for mutating C to A in prokaryotes, a base editing system for mutating C to G in eukaryotes and applications thereof. The base editing system for mutating C to A of the present invention includes cytosine deaminase AID and nCas9 nuclease or includes cytosine deaminase AID, nCas9 nuclease and uracil DNA glycosidase; the base editing system for mutating C to G of the present invention includes cytosine deaminase AID and nCas9 nuclease or includes cytosine deaminase APOBEC, nCas9 nuclease and uracil DNA glycosidase. The experiments prove that a combination of the three base editing systems for mutating C to A, C to T and A to G can realize a mutation of A, T, C or G to any base in prokaryotes (such as Escherichia coil); a combination of the three base editing systems for mutating C to G, C to T and A to G can realize a mutation from A, T, C or G to any base in eukaryotes (such as mammalian cells).

1-39. (canceled)

40. A method for mutating a target base C to A in a genome sequence, is D1) or D2) or D3) or D4) as follows:
D1) the method includes the following steps: using a CRISPR/Cas9 system, cytosine deaminase and uracil DNA glycosidase for single-base editing to mutate a target base C to A;
D2) the method includes the following steps: using a CRISPR/Cas9 system and cytosine deaminase for single-base editing to mutate a target base C to A;
D3) the method includes the following steps: using a CRISPR/Cas9 system, cytosine deaminase AID and uracil DNA glycosidase for single-base editing to mutate a target base C to A;
D4) the method includes the following steps: using a CRISPR/Cas9 system and cytosine deaminase AID for single-base editing to mutate a target base C to A.

41. The method according to claim 40, wherein the method is d1) or d2) or d3) or d4) as follows:
d1) the method includes the following steps: introducing a coding gene of cytosine deaminase, a coding gene of CRISPR nuclease, a coding gene of uracil DNA glycosidase and a coding sequence of sgRNA into a receptor organism or the cells of a receptor organism, so that the coding gene of cytosine deaminase, the coding gene of CRISPR nuclease, the coding gene of uracil DNA glycosidase and the coding sequence of sgRNA are all expressed to mutate a target base C to A;
d2) the method includes the following steps: introducing a coding gene of cytosine deaminase, a coding gene of CRISPR nuclease and a coding sequence of sgRNA into a receptor organism or the cells of a receptor organism, so that the coding gene of cytosine deaminase, the coding gene of CRISPR nuclease and the coding sequence of sgRNA are all expressed to mutate a target base C to A;
d3) the method includes the following steps: introducing a coding gene of cytosine deaminase AID, a coding gene of nCas9 nuclease, a coding gene of uracil DNA glycosidase and a coding sequence of sgRNA into a receptor organism or the cells of a receptor organism, so that the coding gene of cytosine deaminase AID, the coding gene of nCas9 nuclease, the coding gene of uracil DNA glycosidase and the coding sequence of sgRNA are all expressed to mutate a target base C to A;
d4) the method includes the following steps: introducing a coding gene of cytosine deaminase AID, a coding gene of nCas9 nuclease and a coding sequence of sgRNA into a receptor organism or the cells of a receptor organism, so that the coding gene of cytosine deaminase AID, the coding gene of nCas9 nuclease and the coding sequence of sgRNA are all expressed to mutate a target base C to A;
The sgRNA targets a target sequence; the target base C locates in the target sequence.

42. The method according to claim 40, wherein the cytosine deaminase or the cytosine deaminase AID is cytosine deaminase pmCDA; or
wherein the uracil DNA glycosidase is an uracil DNA glycosidase ung derived from Escherichia coli; or
wherein the CRISPR nuclease or the nCas9 nuclease is a mutant nCas9-D10A of Cas9.

43. The method according to claim 40, wherein in the d3), the coding gene of the cytosine deaminase AID, the coding gene of the nCas9 nuclease and the coding gene of the uracil DNA glycosidase are introduced into the receptor organism or the cell of the receptor organism through a recombinant plasmid A;
the recombinant plasmid A expresses a fusion protein composed of cytosine deaminase AID, nCas9 nuclease and uracil DNA glycosidase; or
wherein in the d4), the coding gene of the cytosine deaminase AID and the coding gene of the nCas9 nuclease are introduced into the receptor organism or the cell of the receptor organism through a recombinant plasmid B;
the recombinant plasmid B expresses a fusion protein composed of cytosine deaminase AID and nCas9 nuclease.

44. The method according to claim 43, wherein the nucleotide sequence of the recombinant plasmid B is as shown in sequence 1; or
wherein the nucleotide sequence of the recombinant plasmid A is shown in SEQ ID NO:3.

45. The method according to claim 40, wherein the receptor organism is prokaryotes,
preferably, wherein the prokaryote is Escherichia coli;
further preferably, wherein the Escherichia coli is Escherichia coli MG1655 or Escherichia coli ATCC 8739.

46. A method for improving the base editing efficiency of mutating a target base C to G in a genome sequence is E1) or E2) as follows:
E1) the method includes the following steps: using a CRISPR/Cas9 system, cytosine deaminase and uracil DNA glycosidase for single-base editing to improve the base editing efficiency of mutating a target base C to G;
E2) the method includes the following steps: using a CRISPR/Cas9 system, cytosine deaminase APOBEC and uracil DNA glycosidase for single-base editing to improve the base editing efficiency of mutating a target base C to G.

47. The method according to claim 46, wherein the method is e1) or e2) as follows:
e1) the method includes the following steps: introducing a coding gene of cytosine deaminase, a coding gene of CRISPR nuclease, a coding gene of uracil DNA glycosidase and a coding sequence of sgRNA into a receptor organism or the cells of a receptor organism, so that the coding gene of cytosine deaminase, the coding gene of CRISPR nuclease, the coding gene of uracil DNA glycosidase and the coding sequence of sgRNA are all expressed to improve a base editing efficiency of mutating a target base C to G in a genome sequence;
e2) the method includes the following steps: introducing a coding gene of cytosine deaminase APOBEC, a coding gene of nCas9 nuclease, a coding gene of uracil DNA glycosidase and a coding sequence of sgRNA into a receptor organism or the cells of a receptor organism, so that the coding gene of cytosine deaminase APOBEC, the coding gene of nCas9 nuclease, the coding gene of uracil DNA glycosidase and the coding sequence of sgRNA are all expressed to improve a base editing efficiency of mutating a target base C to G in a genome sequence;
The sgRNA targets a target sequence, and the target base locates in the target sequence.

48. The method according to claim 46, wherein the cytosine deaminase or the cytosine deaminase APOBEC, is cytosine deaminase APOBEC1; or
wherein the uracil DNA glycosidase is a protein represented by an amino acid sequence obtained by deleting the amino acid sequence shown at sites 1 to 84 of the human-derived uracil DNA glycosidase UNG amino acid sequence from the N-terminal; or
wherein the CRISPR nuclease or the nCas9 nuclease is a Cas9 mutant nCas9-D10A.

49. The method according to claim 46, wherein in e2), the coding gene of the cytosine deaminase APOBEC, the coding gene of the nCas9 nuclease and the coding gene of the uracil DNA glycosidase are introduced into the receptor organism or the cells of the receptor organism through a recombinant plasmid C;
the recombinant plasmid C expresses a fusion protein composed of cytosine deaminase APOBEC, nCas9 nuclease and uracil DNA glycosidase.

50. The method according to claim 49, wherein the nucleotide sequence of the recombinant plasmid C is as shown in SEQ ID NO: 5.

51. The method according to claim 46, wherein the receptor biological cells are eukaryotic cells,
preferably, wherein the eukaryotic cells are mammalian cells.

52. A method for realizing a site-directed mutation from any base to any base in a genome sequence in prokaryotes is M1) or M2) or M3) or M4) as follows:
M1 includes m1) or m2) or m3):
m1) when a target base in a genome sequence is a base C, starting from the base C, the target base can be mutated from the base C to a base T using a base editing system for mutating C to T, so as to realize the editing from the base C to the base T;
m2) when a target base in a genome sequence is a base C, starting from the base C, the target base can be mutated from the base C to a base A using a base editing system for mutating C to A, so as to realize the editing from the base C to the base A;
m3) when a target base in a genome sequence is a base C, a mutant taking a base A as the target base is obtained according to the method described in m2); starting from the base A, the target base can be mutated from the base A to a base G using a base editing system for mutating A to G, so as to realize the editing from the base C to the base G;
any site-directed mutation from the base C to the base T, the base A and the base G is therefore realized;
M2) when a target base in a genome sequence is a base G, since the base G is a complementary base of a base C, any site-directed mutation from the base G to the base A, the base T and the base C is also realized according to the method described in M1);
M3 includes m4) or m5) or m6):
m4) when a target base in a genome sequence is a base T, a base A is a complementary base of the target base; starting from the base A, the complementary base of the target base can be mutated from the base A to a base G using a base editing system for mutating A to G, so as to realize the editing from the base T to the base G;
m5) when a target base in a genome sequence is a base T, a mutant taking a base C as the target base is obtained according to the method described in m4); starting from the base C, the target base can be mutated from the base C to a base A using a base editing system for mutating C to A, so as to realize the editing from the base T to the base A;
m6) when a target base in a genome sequence is a base T, a mutant taking a base A as the target base is obtained according to the method described in m5); starting from the base A, the target base can be mutated from the base A to a base G using a base editing system for mutating A to G, so as to realize the editing from the base T to the base G;
any site-directed mutation from the base T to the base C, the base A and the base G is therefore realized;
M4) when a target base in a genome sequence is a base A, since the base A is a complementary base of a base T, any site-directed mutation from the base A to the base G, the base T and the base C is also realized according to the method described in M3);
The base editing system for mutating C to A is a base editing system I for mutating C to A, or a base editing system II for mutating C to A, or a base editing system III for mutating C to A, or a base editing system IV for mutating C to A;
The base editing system I for mutating C to A comprises cytosine deaminase or a biomaterial related to the cytosine deaminase, CRISPR nuclease or a biomaterial related to the CRISPR nuclease, and uracil DNA glycosidase or a biomaterial related to the uracil DNA glycosidase;
The base editing system II for mutating C to A comprises cytosine deaminase or a biomaterial related to the cytosine deaminase, and CRISPR nuclease or a biomaterial related to the CRISPR nuclease;
The base base editing system III for mutating C to A comprises cytosine deaminase AID or a biomaterial related to the cytosine deaminase AID, nCas9 nuclease or a biomaterial related to the nCas9 nuclease and uracil DNA glycosidase or a biomaterial related to the uracil DNA glycosidase;
The base editing system IV for mutating C to A comprises cytosine deaminase AID or a biomaterial related to the cytosine deaminase AID, and nCas9 nuclease or a biomaterial related to the nCas9 nuclease.

53. The method according to claim 52, wherein the cytosine deaminase or the cytosine deaminase AID is cytosine deaminase pmCDA; or
the uracil DNA glycosidase is the uracil DNA glycosidase ung derived from Escherichia coli; or
wherein the CRISPR nuclease or the nCas9 nuclease is a mutant nCas9-D10A of Cas9.

54. The method according to claim 52, wherein the prokaryote is Escherichia coli;
preferably, wherein the Escherichia coli is Escherichia coli MG1655 or Escherichia coil ATCC 8739.

55. A method for realizing a site-directed mutation from any base to any base in a genome sequence in eukaryotes is N1) or N2) or N3) or N4) as follows:
N1) includes n1) or n2) or n3):
n1) when a target base in a genome sequence is a base C, starting from the base C, the target base can be mutated from the base C to a base T using a base editing system for mutating C to T to realize the base editing from C to T;
n2) when a target base in a genome sequence is a base C, starting from the base C, the target base can be mutated from the base C to a base G using a base editing system for mutating C to G so as to realize the editing from the base C to the base G;
n3) when a target base in a genome sequence is a base C, a mutant taking a base G as the target base is obtained according to the method described in n2), and the base C is a complementary base of the base G; starting from the base C, the target base can be mutated from the base C to a base T using a base editing system for mutating C to T, and a base A is a complementary base of the base T, realizing the editing from the base C to the base A;
any site-directed mutation from the base C to the base T, the base A and the base G is therefore realized;
N2) when a target base in a genome sequence is a base G, since the base G is a complementary base of a base C, any site-directed mutation from the base G to the base A, the base T and the base C is also realized according to the method described in N1);
N3 includes n4) or n5) or n6):
n4) when a target base in a genome sequence is a base T, a base A is a complementary base of the base T; starting from the base A, the complementary base of the target base can be mutated from the base A to a base G using a base editing system for mutating A to G so as to realize the editing from the base T to the base G;
n5) when a target base in a genome sequence is a base T, a mutant taking a base C as the target base is obtained according to the method described in n4); starting from the base C, the target base can be mutated from the base C to a base G using a base editing system for mutating C to G so as to realize the editing from the base T to the base G;
n6) when a target base in a genome sequence is a base T, a mutant taking a base G as the target base is obtained according to the method described in n5), and a base C is a complementary base of the base G; starting from the base C, the complementary base of the target base can be mutated from the base C to a base T using a base editing system for mutating C to T so as to realize the editing from the base T to the base A;
any site-directed mutation from the base T to the base C, the base A and the base G is therefore realized;
N4) when a target base in a genome sequence is a base A, since the base A is a complementary base of a base T, any site-directed mutation from the base A to the base G, the base T and the base C is also realized according to the method described in N3);
The base editing system for mutating C to G is a base editing system I for mutating C to G, or a base editing system II for mutating C to G, or a base editing system III for mutating C to G, or a base editing system IV for mutating C to G;
The base editing system I for mutating C to G comprises cytosine deaminase or a biomaterial related to the cytosine deaminase, CRISPR nuclease or a biomaterial related to the CRISPR nuclease, and uracil DNA glycosidase or a biomaterial related to the uracil DNA glycosidase;
The base editing system II for mutating C to G comprises cytosine deaminase or a biomaterial related to the cytosine deaminase, and CRISPR nuclease or a biomaterial related to the CRISPR nuclease;
The base editing system III for mutating C to G comprises cytosine deaminase APOBEC or a biomaterial related to the cytosine deaminase APOBEC, nCas9 nuclease or a biomaterial related to the nCas9 nuclease and uracil DNA glycosidase or a biomaterial related to the uracil DNA glycosidase;
The base editing system IV for mutating C to G comprises cytosine deaminase APOBEC or a biomaterial related to the cytosine deaminase APOBEC, and nCas9 nuclease or a biomaterial related to the nCas9 nuclease.

56. The method according to claim 55, wherein the cytosine deaminase or the cytosine deaminase APOBEC is the cytosine deaminase APOBEC1; or
wherein the uracil DNA glycosidase is a protein represented by an amino acid sequence obtained by deleting the amino acid sequence represented by positions 1 to 84 of the human uracil DNA glycosidase UNG amino acid sequence from the N-terminal; or
wherein the CRISPR nuclease or the nCas9 nuclease is a Cas9 mutant nCas9-D10A.

57. The method according to claim 56, wherein the is eukaryotic cells,
preferably, wherein the eukaryotic cells are mammalian cells.

58. A product comprising: any of the following products described in c1)-c5):
c1) a product for mutating a target base C to A in a genome sequence, including the base editing system I for mutating C to A described in claim 52, or the base editing system II for mutating C to A, or the base editing system III for mutating C to A, or the base editing system IV for mutating C to A;
c2) a product for improving a base editing efficiency of mutating a target base C to A in a genome sequence, including the base editing system I for mutating C to A, or the base base editing system III for mutating C to A;
c3) a product for improving a base editing efficiency of mutating a target base C to G in a genome sequence, including the base editing system I for mutating C to G, or the base editing system III for mutating C to G;
c4) a product for realizing a site-directed mutation from any base to any base in a genome sequence in prokaryotes, including a base editing system for mutating C to A, a base editing system for mutating C to T, and a base editing system for mutating A to G;
wherein the base editing system for mutating C to A is the base editing system I for mutating C to A, or the base editing system II for mutating C to A, or the base editing system III for mutating C to A, or the base editing system IV for mutating C to A;
c5) a product for realizing a site-directed mutation from any base to any base in a genome sequence in eukaryotes, including a base editing system for mutating C to G, a base editing system for mutating C to T, and a base editing system for mutating A to G;
wherein the base editing system for mutating C to G is the base editing system I for mutating C to G, or the base editing system II for mutating C to G or the base editing system III for mutating C to G, or the base editing system IV for mutating C to G.


SEQUENCE LISTING (269 sequences)

SEQ ID NO: 1 - DNA, 9027 bp, Artificial Sequence
  Synthesized
atggataagaaatactcaataggcttagctatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgacccaaagaagaaacgcaaggtcggtggtggtggctctggcggtggcggttctgcagaatacgtgcgcgccctgttcgactttaacggcaacgatgaggaggatctgccattcaagaagggcgatatcctccgcatccgtgataagcctgaggaacagtggtggaacgccgaagactccgaaggtaagcgcggcatgatccctgtgccatacgtcgagaagtactccggcgattacaaggaccatgatggcgattataaggatcacgacatcgactataaggacgacgacgataaatcccgcatgaccgatgcggagtacgttcgtatccacgaaaagctggatatctacaccttcaagaagcagttctttaacaacaagaaaagcgtgagccaccgttgctacgttctgttcgagctgaaacgtcgtggtgaacgtcgtgcgtgcttttggggctatgcggtgaacaagccgcaaagcggtaccgagcgtggcatccacgcggaaatcttcagcattcgtaaagttgaggaatacctgcgtgacaacccgggccagtttaccatcaactggtatagcagctggagcccgtgcgcggattgcgcggagaagattctggaatggtacaaccaagagctgcgtggtaacggccacaccctgaagatctgggcgtgcaaactgtactatgaaaagaacgcgcgtaaccagattggcctgtggaacctgcgtgacaacggtgtgggcctgaacgtgatggttagcgagcactatcaatgctgccgtaaaatctttattcagagcagccacaaccaactgaacgagaaccgttggctggaaaagaccctgaaacgtgcggagaagcgtcgtagcgaactgagcatcatgattcaagtgaaaatcctgcacaccaccaagagcccggcggttagccgtggtagcggctaagtgcctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctttaattaacgatgataagctgtcaaacatgagaattacaacttatatcgtatggggctgacttcaggtgctacatttgaagagataaattgcactgaaatctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctcccttaattaaactagtcatatgggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctggtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatggaattcaaggagatatacc

SEQ ID NO: 2 - DNA, 2681 bp, Artificial Sequence
  Synthesized
aaatttaattaagtgtaggctggagctgcttcgaagttcctatactttctagagaataggaacttcggaataggaacttcaagatcccctcacgctgccgcaagcacgtgatcgaaatccagatccttgacccgcagttgcaaaccctcactgatccggctcacggtaactgatgccgtatttgcagtaccagcgtacggcccacagaatgatgtcacgctgaaaatgccggcctttgaatgggttcatgtgcagctccatcagcaaaaggggatgataagtttatcaccaccgactatttgcaacagtgccgttgatcgtgctatgatcgactgatgtcatcagcggtggagtgcaatgtcgtgcaatacgaatggcgaaaagccgagctcatcggtcagcttctcaaccttggggttacccccggcggtgtgctgctggtccacagctccttccgtagcgtccggcccctcgaagatgggccacttggactgatcgaggccctgcgtgctgcgctgggtccgggagggacgctcgtcatgccctcgtggtcaggtctggacgacgagccgttcgatcctgccacgtcgcccgttacaccggaccttggagttgtctctgacacattctggcgcctgccaaatgtaaagcgcagcgcccatccatttgcctttgcggcagcggggccacaggcagagcagatcatctctgatccattgcccctgccacctcactcgcctgcaagcccggtcgcccgtgtccatgaactcgatgggcaggtacttctcctcggcgtgggacacgatgccaacacgacgctgcatcttgccgagttgatggcaaaggttccctatggggtgccgagacactgcaccattcttcaggatggcaagttggtacgcgtcgattatctcgagaatgaccactgctgtgagcgctttgccttggcggacaggtggctcaaggagaagagccttcagaaggaaggtccagtcggtcatgcctttgctcggttgatccgctcccgcgacattgtggcgacagccctgggtcaactgggccgagatccgttgatcttcctgcatccgccagagggcgggatgcgaagaatgcgatgccgctcgccagtcgattggctgagctcatgagcggagaacgagatgacgttggaggggcaaggtcgcgctgattgctggggcaacacgtgaaaggcgagatcaccaaggtagtcggcaaataatgtctaacaattcgttcaagccgacgccgcttcgcggcgcggcttaactcaagcgttagatgcactaagcacataattgctcacagccaaactatcaggtcaagtctgctctagcacctgaagtcagcctgtctaggtttatacataggcgagtactctgttatggagtcagatcttagctcccgcccggtgcagtatgagttttagagctagaaatagcaagttaaaataaggctagtccgttatcaacttgaaaaagtggcaccgagtcggtgcttagcatccaaactcgagtaaggatctccaggcatcaaataaaacgaaaggctcagtcgaaagactgggcctttcgttttatctgttgtttgtcggtgaacgctctctactagagtcacactggctcaccttcgggtgggcctttctgcgtttatacctagggcgttcggctgcggctctacttttgtttgttagtcttgatgcttcactgatagatacaagagccataagaacctcagatccttccgtatttagccagtatgttctctagtgtggttcgttgagcgacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgattt

SEQ ID NO: 3 - DNA, 9762 bp, Artificial Sequence
  Synthesized
atggctaacgaattaacctggcatgacgtgctggctgaagagaagcagcaaccctattttcttaatacccttcagaccgtcgccagcgagcggcagtccggcgtcactatctacccaccacaaaaagatgtctttaacgcgttccgctttacagagttgggtgacgttaaagtggtgattctcggccaggatccttatcacggaccgggacaggcgcatggtctggcattttccgttcgtcccggcattgccattcctccgtcattattgaatatgtataaagagctggaaaatactattccgggcttcacccgccctaatcatggttatcttgaaagctgggcgcgtcagggcgttctgctactcaatactgtgttgacggtacgcgcaggtcaggcgcattcccacgccagcctcggctgggaaaccttcaccgataaagtgatcagcctgattaaccagcatcgcgaaggcgtggtgtttttgttgtggggatcgcatgcgcaaaagaaaggggcgattatagataagcaacgccatcatgtactgaaagcaccgcatccgtcgccgctttcggcgcatcgtggattctttggctgcaaccattttgtgctggcaaatcagtggctggaacaacgtggcgagacgccgattgactggatgccagtattaccggcagagagtgagagcggtagcgaaaccccgggtaccagcgagagcgcgaccccggaaagcatggataagaaatactcaataggcttagctatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgacccaaagaagaaacgcaaggtcggtggtggtggctctggcggtggcggttctgcagaatacgtgcgcgccctgttcgactttaacggcaacgatgaggaggatctgccattcaagaagggcgatatcctccgcatccgtgataagcctgaggaacagtggtggaacgccgaagactccgaaggtaagcgcggcatgatccctgtgccatacgtcgagaagtactccggcgattacaaggaccatgatggcgattataaggatcacgacatcgactataaggacgacgacgataaatcccgcatgaccgatgcggagtacgttcgtatccacgaaaagctggatatctacaccttcaagaagcagttctttaacaacaagaaaagcgtgagccaccgttgctacgttctgttcgagctgaaacgtcgtggtgaacgtcgtgcgtgcttttggggctatgcggtgaacaagccgcaaagcggtaccgagcgtggcatccacgcggaaatcttcagcattcgtaaagttgaggaatacctgcgtgacaacccgggccagtttaccatcaactggtatagcagctggagcccgtgcgcggattgcgcggagaagattctggaatggtacaaccaagagctgcgtggtaacggccacaccctgaagatctgggcgtgcaaactgtactatgaaaagaacgcgcgtaaccagattggcctgtggaacctgcgtgacaacggtgtgggcctgaacgtgatggttagcgagcactatcaatgctgccgtaaaatctttattcagagcagccacaaccaactgaacgagaaccgttggctggaaaagaccctgaaacgtgcggagaagcgtcgtagcgaactgagcatcatgattcaagtgaaaatcctgcacaccaccaagagcccggcggttagccgtggtagcggctaagtgcctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctttaattaacgatgataagctgtcaaacatgagaattacaacttatatcgtatggggctgacttcaggtgctacatttgaagagataaattgcactgaaatctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctcccttaattaaactagtcatatgggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctggtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatggaattcaaggagatatacc

SEQ ID NO: 4 - DNA, 9185 bp, Artificial Sequence
  Synthesized
gccaagctaattcgagctcggtacctgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgtcgacggatccatgccaaagaagaagaggaaggtttcatcggagaccggccctgttgctgttgaccccaccctgcggcggagaatcgagccacacgagttcgaggtgttcttcgacccaagggagctccgcaaggagacgtgcctcctgtacgagatcaactggggcggcaggcactccatctggaggcacaccagccaaaacaccaacaagcacgtggaggtcaacttcatcgagaagttcaccaccgagaggtacttctgcccaaacacccgctgctccatcacctggttcctgtcctggagcccatgcggcgagtgctccagggccatcaccgagttcctcagccgctacccacacgtcaccctgttcatctacatcgccaggctctaccaccacgccgacccaaggaacaggcagggcctccgcgacctgatctccagcggcgtgaccatccaaatcatgaccgagcaggagtccggctactgctggaggaacttcgtcaactactccccaagcaacgaggcccactggccaaggtacccacacctctgggtgcgcctctacgtgctcgagctgtactgcatcatcctcggcctgccaccatgcctcaacatcctgaggcgcaagcaaccacagctgaccttcttcaccatcgccctccaaagctgccactaccagaggctcccaccacacatcctgtgggctaccggcctcaagtccggcagcgagacgccaggcacctccgagagcgctacgcctgaacttaaggacaagaagtactcgatcggcctcgccatcgggacgaactcagttggctgggccgtgatcaccgacgagtacaaggtgccctctaagaagttcaaggtcctggggaacaccgaccgccattccatcaagaagaacctcatcggcgctctcctgttcgacagcggggagaccgctgaggctacgaggctcaagagaaccgctaggcgccggtacacgagaaggaagaacaggatctgctacctccaagagattttctccaacgagatggccaaggttgacgattcattcttccaccgcctggaggagtctttcctcgtggaggaggataagaagcacgagcggcatcccatcttcggcaacatcgtggacgaggttgcctaccacgagaagtaccctacgatctaccatctgcggaagaagctcgtggactccaccgataaggcggacctcagactgatctacctcgctctggcccacatgatcaagttccgcggccatttcctgatcgagggggatctcaacccagacaacagcgatgttgacaagctgttcatccaactcgtgcagacctacaaccaactcttcgaggagaacccgatcaacgcctctggcgtggacgcgaaggctatcctgtccgcgaggctctcgaagtccaggaggctggagaacctgatcgctcagctcccaggcgagaagaagaacggcctgttcgggaacctcatcgctctcagcctggggctcaccccgaacttcaagtcgaacttcgatctcgctgaggacgccaagctgcaactctccaaggacacctacgacgatgacctcgataacctcctggcccagatcggcgatcaatacgcggacctgttcctcgctgccaagaacctgtcggacgccatcctcctgtcagatatcctccgcgtgaacaccgagatcacgaaggctccactctctgcctccatgatcaagcgctacgacgagcaccatcaggatctgaccctcctgaaggcgctggtccgccaacagctcccggagaagtacaaggagattttcttcgatcagtcgaagaacggctacgctgggtacatcgacggcggggcctcacaagaggagttctacaagttcatcaagccaatcctggagaagatggacggcacggaggagctcctggtgaagctcaacagggaggacctcctgcggaagcagagaaccttcgataacggcagcatcccccaccaaatccatctcggggagctgcacgccatcctgagaaggcaagaggacttctaccctttcctcaaggataaccgggagaagatcgagaagatcctgaccttcagaatcccatactacgtcggccctctcgcgcgggggaactcaagattcgcttggatgacccgcaagtctgaggagaccatcacgccgtggaacttcgaggaggtggtggacaagggcgctagcgctcagtcgttcatcgagaggatgaccaacttcgacaagaacctgcccaacgagaaggtgctccctaagcactcgctcctgtacgagtacttcaccgtctacaacgagctcacgaaggtgaagtacgtcaccgagggcatgcgcaagccagcgttcctgtccggggagcagaagaaggctatcgtggacctcctgttcaagaccaaccggaaggtcacggttaagcaactcaaggaggactacttcaagaagatcgagtgcttcgattcggtcgagatcagcggcgttgaggaccgcttcaacgccagcctcgggacctaccacgatctcctgaagatcatcaaggataaggacttcctggacaacgaggagaacgaggatatcctggaggacatcgtgctgaccctcacgctgttcgaggacagggagatgatcgaggagcgcctgaagacgtacgcccatctcttcgatgacaaggtcatgaagcaactcaagcgccggagatacaccggctgggggaggctgtcccgcaagctcatcaacggcatccgggacaagcagtccgggaagaccatcctcgacttcctcaagagcgatggcttcgccaacaggaacttcatgcaactgatccacgatgacagcctcaccttcaaggaggatatccaaaaggctcaagtgagcggccagggggactcgctgcacgagcatatcgcgaacctcgctggctcccccgcgatcaagaagggcatcctccagaccgtgaaggttgtggacgagctcgtgaaggtcatgggccggcacaagcctgagaacatcgtcatcgagatggccagagagaaccaaaccacgcagaaggggcaaaagaactctagggagcgcatgaagcgcatcgaggagggcatcaaggagctggggtcccaaatcctcaaggagcacccagtggagaacacccaactgcagaacgagaagctctacctgtactacctccagaacggcagggatatgtacgtggaccaagagctggatatcaaccgcctcagcgattacgacgtcgatcatatcgttccccagtctttcctgaaggatgactccatcgacaacaaggtcctcaccaggtcggacaagaaccgcggcaagtcagataacgttccatctgaggaggtcgttaagaagatgaagaactactggaggcagctcctgaacgccaagctgatcacgcaaaggaagttcgacaacctcaccaaggctgagagaggcgggctctcagagctggacaaggccggcttcatcaagcggcagctggtcgagaccagacaaatcacgaagcacgttgcgcaaatcctcgactctcggatgaacacgaagtacgatgagaacgacaagctgatcagggaggttaaggtgatcaccctgaagtctaagctcgtctccgacttcaggaaggatttccagttctacaaggttcgcgagatcaacaactaccaccatgcccatgacgcttacctcaacgctgtggtcggcaccgctctgatcaagaagtacccaaagctggagtccgagttcgtgtacggggactacaaggtttacgatgtgcgcaagatgatcgccaagtcggagcaagagatcggcaaggctaccgccaagtacttcttctactcaaacatcatgaacttcttcaagaccgagatcacgctggccaacggcgagatccggaagagaccgctcatcgagaccaacggcgagacgggggagatcgtgtgggacaagggcagggatttcgcgaccgtccgcaaggttctctccatgccccaggtgaacatcgtcaagaagaccgaggtccaaacgggcgggttctcaaaggagtctatcctgcctaagcggaacagcgacaagctcatcgccagaaagaaggactgggacccaaagaagtacggcgggttcgacagccctaccgtggcctactcggtcctggttgtggcgaaggttgagaagggcaagtccaagaagctcaagagcgtgaaggagctcctggggatcaccatcatggagaggtccagcttcgagaagaacccaatcgacttcctggaggccaagggctacaaggaggtgaagaaggacctgatcatcaagctcccgaagtactctctcttcgagctggagaacggcaggaagagaatgctggcttccgctggcgagctccagaaggggaacgagctcgcgctgccaagcaagtacgtgaacttcctctacctggcttcccactacgagaagctcaagggcagcccggaggacaacgagcaaaagcagctgttcgtcgagcagcacaagcattacctcgacgagatcatcgagcaaatctccgagttcagcaagcgcgtgatcctcgccgacgcgaacctggataaggtcctctccgcctacaacaagcaccgggacaagcccatcagagagcaagcggagaacatcatccatctcttcaccctgacgaacctcggcgctcctgctgctttcaagtacttcgacaccacgatcgatcggaagagatacacctccacgaaggaggtcctggacgcgaccctcatccaccagtcgatcaccggcctgtacgagacgaggatcgacctctcacaactcggcggggataagagacccgcagcaaccaagaaggcagggcaagcaaagaagaagaagacgcgtgactccggcggcagcaccaacctgtccgacatcatcgagaaggagacgggcaagcaactcgtgatccaggagagcatcctcatgctgccagaggaggtggaggaggtcatcggcaacaagccagagtccgacatcctggtgcacaccgcctacgacgagtccaccgacgagaacgtcatgctcctgaccagcgacgccccagagtacaagccatgggccctcgtcatccaggacagcaacggggagaacaagatcaagatgctgtcgggggggagcccaaagaagaagcggaaggtgtagtgtacaagtaacccgggaattcggtacgctgaaatcaccagtctctctctacaaatctatctctctctattttctccataaataatgtgtgagtagtttcccgataagggaaattagggttcttatagggtttcgctcatgtgttgagcatataagaaacccttagtatgtatttgtatttgtaaaatacttctatcaataaaatttctaattcctaaaaccaaaatccagtactaaaatccagatctcctaaagtccctatagatctttgtcgtgaatataaaccagacacgagacgactaaacctggagcccagacgccgttcgaagctagaagtaccgcttaggcaggaggccgttagggaaaagatgctaaggcagggttggttacgttgactcccccgtaggtttggtttaaatatgatgaagtggacggaaggaaggaggaagacaaggaaggataaggttgcaggccctgtgcaaggtaagaagatggaaatttgatagaggtacgctactatacttatactatacgctaagggaatgcttgtatttataccctataccccctaataaccccttatcaatttaagaaataatccgcataagcccccgcttaaaaattggtatcagagccatgaataggtctatgaccaaaactcaagaggataaaacctcaccaaaatacgaaagagttcttaactctaaagataaaagatctttcaagatcaaaactagttccctcacaccggtgacggggatcgcatgcgatatctcgagatctagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgccagt

SEQ ID NO: 5 - DNA, 9599 bp, Artificial Sequence
  Synthesized
atgtttggagagagctggaagaagcacctcagcggggagttcgggaaaccgtattttatcaagctaatgggatttgttgcagaagaaagaaagcattacactgtttatccacccccacaccaagtcttcacctggacccagatgtgtgacataaaagatgtgaaggttgtcatcctgggacaggatccatatcatggacctaatcaagctcacgggctctgctttagtgttcaaaggcctgttccgcctccgcccagtttggagaacatttataaagagttgtctacagacatagaggattttgttcatcctggccatggagatttatctgggtgggccaagcaaggtgttctccttctcaacgctgtcctcacggttcgtgcccatcaagccaactctcataaggagcgaggctgggagcagttcactgatgcagttgtgtcctggctaaatcagaactcgaatggccttgttttcttgctctggggctcttatgctcagaagaagggcagtgccattgataggaagcggcaccatgtactacagacggctcatccctcccctttgtcagtgtatagagggttctttggatgtagacacttttcaaagaccaatgagctgctgcagaagtctggcaagaagcccattgactggaaggagctgtcgggggggagcccaaagaagaagcggaaggtgtagtgtacaagtaacccgggaattcggtacgctgaaatcaccagtctctctctacaaatctatctctctctattttctccataaataatgtgtgagtagtttcccgataagggaaattagggttcttatagggtttcgctcatgtgttgagcatataagaaacccttagtatgtatttgtatttgtaaaatacttctatcaataaaatttctaattcctaaaaccaaaatccagtactaaaatccagatctcctaaagtccctatagatctttgtcgtgaatataaaccagacacgagacgactaaacctggagcccagacgccgttcgaagctagaagtaccgcttaggcaggaggccgttagggaaaagatgctaaggcagggttggttacgttgactcccccgtaggtttggtttaaatatgatgaagtggacggaaggaaggaggaagacaaggaaggataaggttgcaggccctgtgcaaggtaagaagatggaaatttgatagaggtacgctactatacttatactatacgctaagggaatgcttgtatttataccctataccccctaataaccccttatcaatttaagaaataatccgcataagcccccgcttaaaaattggtatcagagccatgaataggtctatgaccaaaactcaagaggataaaacctcaccaaaatacgaaagagttcttaactctaaagataaaagatctttcaagatcaaaactagttccctcacaccggtgacggggatcgcatgcgatatctcgagatctagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcggccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgccagtgccaagctaattcgagctcggtacctgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgtcgacggatccatgccaaagaagaagaggaaggtttcatcggagaccggccctgttgctgttgaccccaccctgcggcggagaatcgagccacacgagttcgaggtgttcttcgacccaagggagctccgcaaggagacgtgcctcctgtacgagatcaactggggcggcaggcactccatctggaggcacaccagccaaaacaccaacaagcacgtggaggtcaacttcatcgagaagttcaccaccgagaggtacttctgcccaaacacccgctgctccatcacctggttcctgtcctggagcccatgcggcgagtgctccagggccatcaccgagttcctcagccgctacccacacgtcaccctgttcatctacatcgccaggctctaccaccacgccgacccaaggaacaggcagggcctccgcgacctgatctccagcggcgtgaccatccaaatcatgaccgagcaggagtccggctactgctggaggaacttcgtcaactactccccaagcaacgaggcccactggccaaggtacccacacctctgggtgcgcctctacgtgctcgagctgtactgcatcatcctcggcctgccaccatgcctcaacatcctgaggcgcaagcaaccacagctgaccttcttcaccatcgccctccaaagctgccactaccagaggctcccaccacacatcctgtgggctaccggcctcaagtccggcagcgagacgccaggcacctccgagagcgctacgcctgaacttaaggacaagaagtactcgatcggcctcgccatcgggacgaactcagttggctgggccgtgatcaccgacgagtacaaggtgccctctaagaagttcaaggtcctggggaacaccgaccgccattccatcaagaagaacctcatcggcgctctcctgttcgacagcggggagaccgctgaggctacgaggctcaagagaaccgctaggcgccggtacacgagaaggaagaacaggatctgctacctccaagagattttctccaacgagatggccaaggttgacgattcattcttccaccgcctggaggagtctttcctcgtggaggaggataagaagcacgagcggcatcccatcttcggcaacatcgtggacgaggttgcctaccacgagaagtaccctacgatctaccatctgcggaagaagctcgtggactccaccgataaggcggacctcagactgatctacctcgctctggcccacatgatcaagttccgcggccatttcctgatcgagggggatctcaacccagacaacagcgatgttgacaagctgttcatccaactcgtgcagacctacaaccaactcttcgaggagaacccgatcaacgcctctggcgtggacgcgaaggctatcctgtccgcgaggctctcgaagtccaggaggctggagaacctgatcgctcagctcccaggcgagaagaagaacggcctgttcgggaacctcatcgctctcagcctggggctcaccccgaacttcaagtcgaacttcgatctcgctgaggacgccaagctgcaactctccaaggacacctacgacgatgacctcgataacctcctggcccagatcggcgatcaatacgcggacctgttcctcgctgccaagaacctgtcggacgccatcctcctgtcagatatcctccgcgtgaacaccgagatcacgaaggctccactctctgcctccatgatcaagcgctacgacgagcaccatcaggatctgaccctcctgaaggcgctggtccgccaacagctcccggagaagtacaaggagattttcttcgatcagtcgaagaacggctacgctgggtacatcgacggcggggcctcacaagaggagttctacaagttcatcaagccaatcctggagaagatggacggcacggaggagctcctggtgaagctcaacagggaggacctcctgcggaagcagagaaccttcgataacggcagcatcccccaccaaatccatctcggggagctgcacgccatcctgagaaggcaagaggacttctaccctttcctcaaggataaccgggagaagatcgagaagatcctgaccttcagaatcccatactacgtcggccctctcgcgcgggggaactcaagattcgcttggatgacccgcaagtctgaggagaccatcacgccgtggaacttcgaggaggtggtggacaagggcgctagcgctcagtcgttcatcgagaggatgaccaacttcgacaagaacctgcccaacgagaaggtgctccctaagcactcgctcctgtacgagtacttcaccgtctacaacgagctcacgaaggtgaagtacgtcaccgagggcatgcgcaagccagcgttcctgtccggggagcagaagaaggctatcgtggacctcctgttcaagaccaaccggaaggtcacggttaagcaactcaaggaggactacttcaagaagatcgagtgcttcgattcggtcgagatcagcggcgttgaggaccgcttcaacgccagcctcgggacctaccacgatctcctgaagatcatcaaggataaggacttcctggacaacgaggagaacgaggatatcctggaggacatcgtgctgaccctcacgctgttcgaggacagggagatgatcgaggagcgcctgaagacgtacgcccatctcttcgatgacaaggtcatgaagcaactcaagcgccggagatacaccggctgggggaggctgtcccgcaagctcatcaacggcatccgggacaagcagtccgggaagaccatcctcgacttcctcaagagcgatggcttcgccaacaggaacttcatgcaactgatccacgatgacagcctcaccttcaaggaggatatccaaaaggctcaagtgagcggccagggggactcgctgcacgagcatatcgcgaacctcgctggctcccccgcgatcaagaagggcatcctccagaccgtgaaggttgtggacgagctcgtgaaggtcatgggccggcacaagcctgagaacatcgtcatcgagatggccagagagaaccaaaccacgcagaaggggcaaaagaactctagggagcgcatgaagcgcatcgaggagggcatcaaggagctggggtcccaaatcctcaaggagcacccagtggagaacacccaactgcagaacgagaagctctacctgtactacctccagaacggcagggatatgtacgtggaccaagagctggatatcaaccgcctcagcgattacgacgtcgatcatatcgttccccagtctttcctgaaggatgactccatcgacaacaaggtcctcaccaggtcggacaagaaccgcggcaagtcagataacgttccatctgaggaggtcgttaagaagatgaagaactactggaggcagctcctgaacgccaagctgatcacgcaaaggaagttcgacaacctcaccaaggctgagagaggcgggctctcagagctggacaaggccggcttcatcaagcggcagctggtcgagaccagacaaatcacgaagcacgttgcgcaaatcctcgactctcggatgaacacgaagtacgatgagaacgacaagctgatcagggaggttaaggtgatcaccctgaagtctaagctcgtctccgacttcaggaaggatttccagttctacaaggttcgcgagatcaacaactaccaccatgcccatgacgcttacctcaacgctgtggtcggcaccgctctgatcaagaagtacccaaagctggagtccgagttcgtgtacggggactacaaggtttacgatgtgcgcaagatgatcgccaagtcggagcaagagatcggcaaggctaccgccaagtacttcttctactcaaacatcatgaacttcttcaagaccgagatcacgctggccaacggcgagatccggaagagaccgctcatcgagaccaacggcgagacgggggagatcgtgtgggacaagggcagggatttcgcgaccgtccgcaaggttctctccatgccccaggtgaacatcgtcaagaagaccgaggtccaaacgggcgggttctcaaaggagtctatcctgcctaagcggaacagcgacaagctcatcgccagaaagaaggactgggacccaaagaagtacggcgggttcgacagccctaccgtggcctactcggtcctggttgtggcgaaggttgagaagggcaagtccaagaagctcaagagcgtgaaggagctcctggggatcaccatcatggagaggtccagcttcgagaagaacccaatcgacttcctggaggccaagggctacaaggaggtgaagaaggacctgatcatcaagctcccgaagtactctctcttcgagctggagaacggcaggaagagaatgctggcttccgctggcgagctccagaaggggaacgagctcgcgctgccaagcaagtacgtgaacttcctctacctggcttcccactacgagaagctcaagggcagcccggaggacaacgagcaaaagcagctgttcgtcgagcagcacaagcattacctcgacgagatcatcgagcaaatctccgagttcagcaagcgcgtgatcctcgccgacgcgaacctggataaggtcctctccgcctacaacaagcaccgggacaagcccatcagagagcaagcggagaacatcatccatctcttcaccctgacgaacctcggcgctcctgctgctttcaagtacttcgacaccacgatcgatcggaagagatacacctccacgaaggaggtcctggacgcgaccctcatccaccagtcgatcaccggcctgtacgagacgaggatcgacctctcacaactcggcggggataagagacccgcagcaaccaagaaggcagggcaagcaaagaagaagaagacgcgtgactccggcggcagc

SEQ ID NO: 6 - DNA, 4950 bp, Artificial Sequence
  Synthesized
ggtaccgattagtgaacggatctcgacggtatcgatcacgagactagcctcgagcggccgcccccttcaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtcatcttagtcattacctggttttagagctagaaatagcaagttaaaataaggctagtccgttatcaacttgaaaaagtggcaccgagtcggtgctttttttaaagaattctcgacctcgagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggccgcggctcgagggggttggggttgcgccttttccaaggcagccctgggtttgcgcagggacgcggctgctctgggcgtggttccgggaaacgcagcggcgccgaccctgggactcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatccaccggagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggaaacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcatcggtacctttaagaccaatgacttacaaggcagctgtagatcttagccactttctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgggacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 7 - DNA, 8731 bp, Artificial Sequence
  Synthesized
gtgcctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctttaattaacgatgataagctgtcaaacatgagaattacaacttatatcgtatggggctgacttcaggtgctacatttgaagagataaattgcactgaaatctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctcccttaattaaactagtcatatgggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctggtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatggaattcaaggagatataccatgagcgaggtggaattcagccacgagtactggatgcgtcacgcgctgaccctggcgaaacgtgcgcgtgacgagcgtgaagtgccggttggtgcggtgctggttctgaacaaccgtgtgatcggcgagggctggaaccgtgcgattggcctgcatgacccgaccgcgcatgcggaaatcatggcgctgcgtcagggtggcctggttatgcaaaactaccgtctgattgatgcgaccctgtatgtgaccttcgaaccgtgcgttatgtgcgcgggtgcgatgatccacagccgtattggtcgtgtggtttttggcgttcgtaacgcgaaaaccggtgcggcgggcagcctgatggatgtgctgcactacccgggtatgaaccaccgtgttgagatcaccgaaggcattctggcggatgagtgcgcggcgctgctgtgctatttctttcgtatgccgcgtcaggtgttcaacgcgcagaagaaagcgcaaagcagcaccgatagcggtagcgaaaccccgggtaccagcgagagcgcgaccccggaaagcatggataagaaatactcaataggcttagctatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatgccattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactaactcgagtaaggatctccaggcatcaaataaaacgaaaggctcagtcgaaagactgggcctttcgttttatctgttgtttgtcggtgaacgctct

SEQ ID NO: 8 - DNA, 19 bp, Artificial Sequence
  Synthesized
tgctggcgatcttcttggg

SEQ ID NO: 9 - DNA, 21 bp, Artificial Sequence
  Synthesized
cccgtgtcatccatctgtacc

SEQ ID NO: 10 - DNA, 20 bp, Artificial Sequence
  Synthesized
aacggatcgctggttatctg

SEQ ID NO: 11 - DNA, 22 bp, Artificial Sequence
  Synthesized
ccggtacggagatgaatttctg

SEQ ID NO: 12 - DNA, 18 bp, Artificial Sequence
  Synthesized
atcggcgcgaatgatatg

SEQ ID NO: 13 - DNA, 18 bp, Artificial Sequence
  Synthesized
atcactagcccaacaagc

SEQ ID NO: 14 - DNA, 20 bp, Artificial Sequence
  Synthesized
cggttatgcccgctacatgg

SEQ ID NO: 15 - DNA, 20 bp, Artificial Sequence
  Synthesized
gggatcgctgttcgcttcac

SEQ ID NO: 16 - DNA, 22 bp, Artificial Sequence
  Synthesized
tcgcgtactggatctgttctgc

SEQ ID NO: 17 - DNA, 22 bp, Artificial Sequence
  Synthesized
gttgccaacaccttcgactacc

SEQ ID NO: 18 - DNA, 18 bp, Artificial Sequence
  Synthesized
aaccagtacgcctatctc

SEQ ID NO: 19 - DNA, 18 bp, Artificial Sequence
  Synthesized
actcagggttctggattg

SEQ ID NO: 20 - DNA, 18 bp, Artificial Sequence
  Synthesized
cctggcctttgagatgag

SEQ ID NO: 21 - DNA, 18 bp, Artificial Sequence
  Synthesized
gttcaggacgctgtagag

SEQ ID NO: 22 - DNA, 18 bp, Artificial Sequence
  Synthesized
agttgcgtgactacctac

SEQ ID NO: 23 - DNA, 18 bp, Artificial Sequence
  Synthesized
agaccagaccgttcatac

SEQ ID NO: 24 - DNA, 19 bp, Artificial Sequence
  Synthesized
cgtctgaatttgacctgag

SEQ ID NO: 25 - DNA, 21 bp, Artificial Sequence
  Synthesized
ccgtcgatattcagccatgtg

SEQ ID NO: 26 - DNA, 22 bp, Artificial Sequence
  Synthesized
ccctcttccgcttagtaacttg

SEQ ID NO: 27 - DNA, 18 bp, Artificial Sequence
  Synthesized
gaagtgttgcgtcgtcag

SEQ ID NO: 28 - DNA, 20 bp, Artificial Sequence
  Synthesized
cctgatcacctcccaaagtc

SEQ ID NO: 29 - DNA, 20 bp, Artificial Sequence
  Synthesized
cctgatcacctcccaaagtc

SEQ ID NO: 30 - DNA, 20 bp, Artificial Sequence
  Synthesized
aagcagattgaaatcaaatc

SEQ ID NO: 31 - DNA, 20 bp, Artificial Sequence
  Synthesized
aagaagattgaaatcaaatc

SEQ ID NO: 32 - DNA, 20 bp, Artificial Sequence
  Synthesized
aagaagattgaaatcaaatc

SEQ ID NO: 33 - DNA, 20 bp, Artificial Sequence
  Synthesized
aagtagattgaaatcaaatc

SEQ ID NO: 34 - DNA, 20 bp, Artificial Sequence
  Synthesized
aagaagattgaaatcaaatc

SEQ ID NO: 35 - DNA, 20 bp, Artificial Sequence
  Synthesized
ccttcagccaggtaaaccac

SEQ ID NO: 36 - DNA, 20 bp, Artificial Sequence
  Synthesized
cattcagacaggtaaaccac

SEQ ID NO: 37 - DNA, 20 bp, Artificial Sequence
  Synthesized
cattaagccaggtaaaccac

SEQ ID NO: 38 - DNA, 20 bp, Artificial Sequence
  Synthesized
ctttcagccaggtaaaccac

SEQ ID NO: 39 - DNA, 20 bp, Artificial Sequence
  Synthesized
tattcagccaggtaaaccac

SEQ ID NO: 40 - DNA, 20 bp, Artificial Sequence
  Synthesized
ctttcagccaggtaaaccac

SEQ ID NO: 41 - DNA, 20 bp, Artificial Sequence
  Synthesized
cattcagccaggtaaaccac

SEQ ID NO: 42 - DNA, 20 bp, Artificial Sequence
  Synthesized
ctttcagccaggtaaaccac

SEQ ID NO: 43 - DNA, 20 bp, Artificial Sequence
  Synthesized
ctttcagccaggtaaaccac

SEQ ID NO: 44 - DNA, 20 bp, Artificial Sequence
  Synthesized
gctcaggggcttagcaccat

SEQ ID NO: 45 - DNA, 20 bp, Artificial Sequence
  Synthesized
gatcaggggcttagcaccat

SEQ ID NO: 46 - DNA, 20 bp, Artificial Sequence
  Synthesized
gatcaggggcttagcaccat

SEQ ID NO: 47 - DNA, 20 bp, Artificial Sequence
  Synthesized
gctaaggggcttagcaccat

SEQ ID NO: 48 - DNA, 20 bp, Artificial Sequence
  Synthesized
gatcaggggcttagcaccat

SEQ ID NO: 49 - DNA, 20 bp, Artificial Sequence
  Synthesized
gttcaggggcttagcaccat

SEQ ID NO: 50 - DNA, 20 bp, Artificial Sequence
  Synthesized
gcagtcagaactgcatctgg

SEQ ID NO: 51 - DNA, 20 bp, Artificial Sequence
  Synthesized
gaagtcagaactgcatctgg

SEQ ID NO: 52 - DNA, 20 bp, Artificial Sequence
  Synthesized
gaagttagaactgcatctgg

SEQ ID NO: 53 - DNA, 20 bp, Artificial Sequence
  Synthesized
gaagtcagaactgcatctgg

SEQ ID NO: 54 - DNA, 20 bp, Artificial Sequence
  Synthesized
gcaacagacgcaggggcatc

SEQ ID NO: 55 - DNA, 20 bp, Artificial Sequence
  Synthesized
gaaacagacgcaggggcatc

SEQ ID NO: 56 - DNA, 20 bp, Artificial Sequence
  Synthesized
gaaacagacgcaggggcatc

SEQ ID NO: 57 - DNA, 20 bp, Artificial Sequence
  Synthesized
gtaacagacgcaggggcatc

SEQ ID NO: 58 - DNA, 20 bp, Artificial Sequence
  Synthesized
gtaacagacgcaggggcatc

SEQ ID NO: 59 - DNA, 20 bp, Artificial Sequence
  Synthesized
cagctttaccttggtgagat

SEQ ID NO: 60 - DNA, 20 bp, Artificial Sequence
  Synthesized
cagatttaccttggtgagat

SEQ ID NO: 61 - DNA, 20 bp, Artificial Sequence
  Synthesized
aagcagattgaaatcaaatc

SEQ ID NO: 62 - DNA, 20 bp, Artificial Sequence
  Synthesized
aagaagattgaaatcaaatc

SEQ ID NO: 63 - DNA, 20 bp, Artificial Sequence
  Synthesized
aagaagattgaaatcaaatc

SEQ ID NO: 64 - DNA, 20 bp, Artificial Sequence
  Synthesized
aagaagattgaaatcaaatc

SEQ ID NO: 65 - DNA, 20 bp, Artificial Sequence
  Synthesized
gctcaggggcttagcaccat

SEQ ID NO: 66 - DNA, 20 bp, Artificial Sequence
  Synthesized
gatcaggggcttagcaccat

SEQ ID NO: 67 - DNA, 20 bp, Artificial Sequence
  Synthesized
gatcaggggcttagcaccat

SEQ ID NO: 68 - DNA, 20 bp, Artificial Sequence
  Synthesized
gatcaggggcttagcaccat

SEQ ID NO: 69 - DNA, 20 bp, Artificial Sequence
  Synthesized
gatcaggggcttagcaccat

SEQ ID NO: 70 - DNA, 20 bp, Artificial Sequence
  Synthesized
gttcaggggcttagcaccat

SEQ ID NO: 71 - DNA, 20 bp, Artificial Sequence
  Synthesized
gatcaggggcttagcaccat

SEQ ID NO: 72 - DNA, 20 bp, Artificial Sequence
  Synthesized
gatcaggggcttagcaccat

SEQ ID NO: 73 - DNA, 20 bp, Artificial Sequence
  Synthesized
gatcaggggcttagcaccat

SEQ ID NO: 74 - DNA, 20 bp, Artificial Sequence
  Synthesized
atttgtcgatccttcccgcc

SEQ ID NO: 75 - DNA, 20 bp, Artificial Sequence
  Synthesized
atatgtagatccttcccgcc

SEQ ID NO: 76 - DNA, 20 bp, Artificial Sequence
  Synthesized
atatgtagatccttcccgcc

SEQ ID NO: 77 - DNA, 20 bp, Artificial Sequence
  Synthesized
atatgtcgatccttcccgcc

SEQ ID NO: 78 - DNA, 20 bp, Artificial Sequence
  Synthesized
atatgtcgatccttcccgcc

SEQ ID NO: 79 - DNA, 20 bp, Artificial Sequence
  Synthesized
atttgtcgatccttcccgcc

SEQ ID NO: 80 - DNA, 20 bp, Artificial Sequence
  Synthesized
atatgttgatccttcccgcc

SEQ ID NO: 81 - DNA, 20 bp, Artificial Sequence
  Synthesized
tcccgcccggtgcagtatga

SEQ ID NO: 82 - DNA, 20 bp, Artificial Sequence
  Synthesized
taatgcccggtgcagtatga

SEQ ID NO: 83 - DNA, 20 bp, Artificial Sequence
  Synthesized
taacgcccggtgcagtatga

SEQ ID NO: 84 - DNA, 20 bp, Artificial Sequence
  Synthesized
ttccgcccggtgcagtatga

SEQ ID NO: 85 - DNA, 20 bp, Artificial Sequence
  Synthesized
ttacgcccggtgcagtatga

SEQ ID NO: 86 - DNA, 20 bp, Artificial Sequence
  Synthesized
ttacgcccggtgcagtatga

SEQ ID NO: 87 - DNA, 20 bp, Artificial Sequence
  Synthesized
ttacgcccggtgcagtatga

SEQ ID NO: 88 - DNA, 20 bp, Artificial Sequence
  Synthesized
ttcagcccggtgcagtatga

SEQ ID NO: 89 - DNA, 20 bp, Artificial Sequence
  Synthesized
tactgcccggtgcagtatga

SEQ ID NO: 90 - DNA, 20 bp, Artificial Sequence
  Synthesized
tttcgcccggtgcagtatga

SEQ ID NO: 91 - DNA, 20 bp, Artificial Sequence
  Synthesized
tttcgcccggtgcagtatga

SEQ ID NO: 92 - DNA, 20 bp, Artificial Sequence
  Synthesized
taccgcccggtgcagtatga

SEQ ID NO: 93 - DNA, 20 bp, Artificial Sequence
  Synthesized
taccgcccggtgcagtatga

SEQ ID NO: 94 - DNA, 20 bp, Artificial Sequence
  Synthesized
taccgcccggtgcagtatga

SEQ ID NO: 95 - DNA, 20 bp, Artificial Sequence
  Synthesized
taccgcccggtgcagtatga

SEQ ID NO: 96 - DNA, 20 bp, Artificial Sequence
  Synthesized
ttttgcccggtgcagtatga

SEQ ID NO: 97 - DNA, 20 bp, Artificial Sequence
  Synthesized
tttcgcccggtgcagtatga

SEQ ID NO: 98 - DNA, 20 bp, Artificial Sequence
  Synthesized
ttacgcccggtgcagtatga

SEQ ID NO: 99 - DNA, 20 bp, Artificial Sequence
  Synthesized
tttcgcccggtgcagtatga

SEQ ID NO: 100 - DNA, 20 bp, Artificial Sequence
  Synthesized
ttacgcccggtgcagtatga

SEQ ID NO: 101 - DNA, 20 bp, Artificial Sequence
  Synthesized
ttacgcccggtgcagtatga

SEQ ID NO: 102 - DNA, 20 bp, Artificial Sequence
  Synthesized
taacgcccggtgcagtatga

SEQ ID NO: 103 - DNA, 20 bp, Artificial Sequence
  Synthesized
ttacgcccggtgcagtatga

SEQ ID NO: 104 - DNA, 20 bp, Artificial Sequence
  Synthesized
ttacgcccggtgcagtatga

SEQ ID NO: 105 - DNA, 20 bp, Artificial Sequence
  Synthesized
tatcgcccggtgcagtatga

SEQ ID NO: 106 - DNA, 20 bp, Artificial Sequence
  Synthesized
ttacgcccggtgcagtatga

SEQ ID NO: 107 - DNA, 20 bp, Artificial Sequence
  Synthesized
ttacgcccggtgcagtatga

SEQ ID NO: 108 - DNA, 20 bp, Artificial Sequence
  Synthesized
tttcgcccggtgcagtatga

SEQ ID NO: 109 - DNA, 20 bp, Artificial Sequence
  Synthesized
tttcgcccggtgcagtatga

SEQ ID NO: 110 - DNA, 20 bp, Artificial Sequence
  Synthesized
ttcagcccggtgcagtatga

SEQ ID NO: 111 - DNA, 20 bp, Artificial Sequence
  Synthesized
tctcgcccggtgcagtatga

SEQ ID NO: 112 - DNA, 20 bp, Artificial Sequence
  Synthesized
taacgcccggtgcagtatga

SEQ ID NO: 113 - DNA, 20 bp, Artificial Sequence
  Synthesized
tttcgcccggtgcagtatga

SEQ ID NO: 114 - DNA, 20 bp, Artificial Sequence
  Synthesized
tactgcccggtgcagtatga

SEQ ID NO: 115 - DNA, 20 bp, Artificial Sequence
  Synthesized
tacagcccggtgcagtatga

SEQ ID NO: 116 - DNA, 20 bp, Artificial Sequence
  Synthesized
tcacgcccggtgcagtatga

SEQ ID NO: 117 - DNA, 20 bp, Artificial Sequence
  Synthesized
ttccgcccggtgcagtatga

SEQ ID NO: 118 - DNA, 20 bp, Artificial Sequence
  Synthesized
ttacgcccggtgcagtatga

SEQ ID NO: 119 - DNA, 20 bp, Artificial Sequence
  Synthesized
ttacgcccggtgcagtatga

SEQ ID NO: 120 - DNA, 20 bp, Artificial Sequence
  Synthesized
tttcgcccggtgcagtatga

SEQ ID NO: 121 - DNA, 20 bp, Artificial Sequence
  Synthesized
ttacgcccggtgcagtatga

SEQ ID NO: 122 - DNA, 20 bp, Artificial Sequence
  Synthesized
taacgcccggtgcagtatga

SEQ ID NO: 123 - DNA, 20 bp, Artificial Sequence
  Synthesized
taccgcccggtgcagtatga

SEQ ID NO: 124 - DNA, 20 bp, Artificial Sequence
  Synthesized
taacgcccggtgcagtatga

SEQ ID NO: 125 - DNA, 20 bp, Artificial Sequence
  Synthesized
tacagcccggtgcagtatga

SEQ ID NO: 126 - DNA, 20 bp, Artificial Sequence
  Synthesized
taccgcccggtgcagtatga

SEQ ID NO: 127 - DNA, 20 bp, Artificial Sequence
  Synthesized
tttctttcacagatgtggat

SEQ ID NO: 128 - DNA, 20 bp, Artificial Sequence
  Synthesized
tttatttcacagatgtggat

SEQ ID NO: 129 - DNA, 20 bp, Artificial Sequence
  Synthesized
tttatttcacagatgtggat

SEQ ID NO: 130 - DNA, 20 bp, Artificial Sequence
  Synthesized
tttttttcacagatgtggat

SEQ ID NO: 131 - DNA, 20 bp, Artificial Sequence
  Synthesized
tttatttcacagatgtggat

SEQ ID NO: 132 - DNA, 20 bp, Artificial Sequence
  Synthesized
tttttttcacagatgtggat

SEQ ID NO: 133 - DNA, 20 bp, Artificial Sequence
  Synthesized
tttatttcacagatgtggat

SEQ ID NO: 134 - DNA, 20 bp, Artificial Sequence
  Synthesized
tttatttcacagatgtggat

SEQ ID NO: 135 - DNA, 20 bp, Artificial Sequence
  Synthesized
tttttttcacagatgtggat

SEQ ID NO: 136 - DNA, 20 bp, Artificial Sequence
  Synthesized
tttatttcacagatgtggat

SEQ ID NO: 137 - DNA, 20 bp, Artificial Sequence
  Synthesized
ttggcggtttcgctaaatac

SEQ ID NO: 138 - DNA, 20 bp, Artificial Sequence
  Synthesized
ttggaggtttcgctaaatac

SEQ ID NO: 139 - DNA, 20 bp, Artificial Sequence
  Synthesized
ttggtggtttcgctaaatac

SEQ ID NO: 140 - DNA, 20 bp, Artificial Sequence
  Synthesized
ttggcggtttcgctaaatac

SEQ ID NO: 141 - DNA, 20 bp, Artificial Sequence
  Synthesized
ttgagaatacgcccacgcga

SEQ ID NO: 142 - DNA, 20 bp, Artificial Sequence
  Synthesized
ttgagaatacgcccacgcga

SEQ ID NO: 143 - DNA, 20 bp, Artificial Sequence
  Synthesized
ttgagaatacgcccacgcga

SEQ ID NO: 144 - DNA, 20 bp, Artificial Sequence
  Synthesized
ttgagaatacgcccacgcga

SEQ ID NO: 145 - DNA, 20 bp, Artificial Sequence
  Synthesized
ttgtgaatacgcccacgcga

SEQ ID NO: 146 - DNA, 20 bp, Artificial Sequence
  Synthesized
ttgagaataagcccacgcga

SEQ ID NO: 147 - DNA, 20 bp, Artificial Sequence
  Synthesized
ttgtgaatacgcccacgcga

SEQ ID NO: 148 - DNA, 20 bp, Artificial Sequence
  Synthesized
tcccgcccggtgcagtatga

SEQ ID NO: 149 - DNA, 20 bp, Artificial Sequence
  Synthesized
taacgcccggtgcagtatga

SEQ ID NO: 150 - DNA, 20 bp, Artificial Sequence
  Synthesized
tagcgcccggtgcagtatga

SEQ ID NO: 151 - DNA, 20 bp, Artificial Sequence
  Synthesized
taacgcccggtgcagtatga

SEQ ID NO: 152 - DNA, 20 bp, Artificial Sequence
  Synthesized
taacgcccggtgcagtatga

SEQ ID NO: 153 - DNA, 20 bp, Artificial Sequence
  Synthesized
taacgcccggtgcagtatga

SEQ ID NO: 154 - DNA, 20 bp, Artificial Sequence
  Synthesized
taacgcccggtgcagtatga

SEQ ID NO: 155 - DNA, 20 bp, Artificial Sequence
  Synthesized
tactgccaggtgcagtatga

SEQ ID NO: 156 - DNA, 20 bp, Artificial Sequence
  Synthesized
taacgcccggtgcagtatga

SEQ ID NO: 157 - DNA, 20 bp, Artificial Sequence
  Synthesized
taacgcccggtgcagtatga

SEQ ID NO: 158 - DNA, 20 bp, Artificial Sequence
  Synthesized
taacgcccggtgcagtatga

SEQ ID NO: 159 - DNA, 20 bp, Artificial Sequence
  Synthesized
taacgcccggtgcagtatga

SEQ ID NO: 160 - DNA, 20 bp, Artificial Sequence
  Synthesized
taaagcccggtgcagtatga

SEQ ID NO: 161 - DNA, 20 bp, Artificial Sequence
  Synthesized
taacgcccggtgcagtatga

SEQ ID NO: 162 - DNA, 20 bp, Artificial Sequence
  Synthesized
taacgcccggtgcagtatga

SEQ ID NO: 163 - DNA, 20 bp, Artificial Sequence
  Synthesized
taacgcccggtgcagtatga

SEQ ID NO: 164 - DNA, 20 bp, Artificial Sequence
  Synthesized
taacgcccggtgcagtatga

SEQ ID NO: 165 - DNA, 20 bp, Artificial Sequence
  Synthesized
taacgccaggtgcagtatga

SEQ ID NO: 166 - DNA, 20 bp, Artificial Sequence
  Synthesized
taacgcccggtgcagtatga

SEQ ID NO: 167 - DNA, 20 bp, Artificial Sequence
  Synthesized
taccgcccggtgcagtatga

SEQ ID NO: 168 - DNA, 20 bp, Artificial Sequence
  Synthesized
taacgccaggtgcagtatga

SEQ ID NO: 169 - DNA, 20 bp, Artificial Sequence
  Synthesized
tatcgcacggtgcagtatga

SEQ ID NO: 170 - DNA, 20 bp, Artificial Sequence
  Synthesized
taacgcccggtgcagtatga

SEQ ID NO: 171 - DNA, 20 bp, Artificial Sequence
  Synthesized
taacgcccggtgcagtatga

SEQ ID NO: 172 - DNA, 20 bp, Artificial Sequence
  Synthesized
taacgcccggtgcagtatga

SEQ ID NO: 173 - DNA, 20 bp, Artificial Sequence
  Synthesized
taacgcccggtgcagtatga

SEQ ID NO: 174 - DNA, 20 bp, Artificial Sequence
  Synthesized
taacgcccggtgcagtatga

SEQ ID NO: 175 - DNA, 20 bp, Artificial Sequence
  Synthesized
taacgcacggtgcagtatga

SEQ ID NO: 176 - DNA, 20 bp, Artificial Sequence
  Synthesized
taacgcccggtgcagtatga

SEQ ID NO: 177 - DNA, 20 bp, Artificial Sequence
  Synthesized
taatgcccggtgcagtatga

SEQ ID NO: 178 - DNA, 20 bp, Artificial Sequence
  Synthesized
taacgcccggtgcagtatga

SEQ ID NO: 179 - DNA, 20 bp, Artificial Sequence
  Synthesized
taacgcccggtgcagtatga

SEQ ID NO: 180 - DNA, 20 bp, Artificial Sequence
  Synthesized
taacgcccggtgcagtatga

SEQ ID NO: 181 - DNA, 20 bp, Artificial Sequence
  Synthesized
taacgcccggtgcagtatga

SEQ ID NO: 182 - DNA, 20 bp, Artificial Sequence
  Synthesized
taacgcccggtgcagtatga

SEQ ID NO: 183 - DNA, 20 bp, Artificial Sequence
  Synthesized
taacgcccggtgcagtatga

SEQ ID NO: 184 - DNA, 20 bp, Artificial Sequence
  Synthesized
taacgcccggtgcagtatga

SEQ ID NO: 185 - DNA, 20 bp, Artificial Sequence
  Synthesized
taacgcccggtgcagtatga

SEQ ID NO: 186 - DNA, 20 bp, Artificial Sequence
  Synthesized
taacgcgcggtgcagtatga

SEQ ID NO: 187 - DNA, 20 bp, Artificial Sequence
  Synthesized
taacgcccggtgcagtatga

SEQ ID NO: 188 - DNA, 20 bp, Artificial Sequence
  Synthesized
atctgtcgatccttcccgcc

SEQ ID NO: 189 - DNA, 20 bp, Artificial Sequence
  Synthesized
atttgtcgatccttcccgcc

SEQ ID NO: 190 - DNA, 20 bp, Artificial Sequence
  Synthesized
atatgtcgatccttcccgcc

SEQ ID NO: 191 - DNA, 20 bp, Artificial Sequence
  Synthesized
atatgtcgatccttcccgcc

SEQ ID NO: 192 - DNA, 20 bp, Artificial Sequence
  Synthesized
atatgtcgatccttcccgcc

SEQ ID NO: 193 - DNA, 20 bp, Artificial Sequence
  Synthesized
atatgtcgatccttcccgcc

SEQ ID NO: 194 - DNA, 20 bp, Artificial Sequence
  Synthesized
atatgtcgatccttcccgcc

SEQ ID NO: 195 - DNA, 20 bp, Artificial Sequence
  Synthesized
atatgtcgatccttcccgcc

SEQ ID NO: 196 - DNA, 20 bp, Artificial Sequence
  Synthesized
atatgtcgatccttcccgcc

SEQ ID NO: 197 - DNA, 20 bp, Artificial Sequence
  Synthesized
atatgtcgatccttcccgcc

SEQ ID NO: 198 - DNA, 20 bp, Artificial Sequence
  Synthesized
atttgtcgatccttcccgcc

SEQ ID NO: 199 - DNA, 20 bp, Artificial Sequence
  Synthesized
atatgtcgatccttcccgcc

SEQ ID NO: 200 - DNA, 20 bp, Artificial Sequence
  Synthesized
atatgtcgatccttcccgcc

SEQ ID NO: 201 - DNA, 20 bp, Artificial Sequence
  Synthesized
atatgtcgatccttcccgcc

SEQ ID NO: 202 - DNA, 20 bp, Artificial Sequence
  Synthesized
ttgcgaatacgcccacgcga

SEQ ID NO: 203 - DNA, 20 bp, Artificial Sequence
  Synthesized
ttgagaatacgcccacgcga

SEQ ID NO: 204 - DNA, 20 bp, Artificial Sequence
  Synthesized
ttgagaatacgcccacgcga

SEQ ID NO: 205 - DNA, 20 bp, Artificial Sequence
  Synthesized
ttgagaatacgcccacgcga

SEQ ID NO: 206 - DNA, 20 bp, Artificial Sequence
  Synthesized
ttgagaatacgcccacgcga

SEQ ID NO: 207 - DNA, 20 bp, Artificial Sequence
  Synthesized
ttgagaatacgcccacgcga

SEQ ID NO: 208 - DNA, 20 bp, Artificial Sequence
  Synthesized
ttgagaatacgcccacgcga

SEQ ID NO: 209 - DNA, 20 bp, Artificial Sequence
  Synthesized
ttgagaatacgcccacgcga

SEQ ID NO: 210 - DNA, 20 bp, Artificial Sequence
  Synthesized
ttgagaatacgcccacgcga

SEQ ID NO: 211 - DNA, 20 bp, Artificial Sequence
  Synthesized
ttgagaatacgcccacgcga

SEQ ID NO: 212 - DNA, 20 bp, Artificial Sequence
  Synthesized
ttgagaatacgcccacgcga

SEQ ID NO: 213 - DNA, 20 bp, Artificial Sequence
  Synthesized
ttgagaatacgcccacgcga

SEQ ID NO: 214 - DNA, 20 bp, Artificial Sequence
  Synthesized
ttgagaatacgcccacgcga

SEQ ID NO: 215 - DNA, 20 bp, Artificial Sequence
  Synthesized
ttgagaatacgcccacgcga

SEQ ID NO: 216 - DNA, 20 bp, Artificial Sequence
  Synthesized
ttgagaatacgcccacgcga

SEQ ID NO: 217 - DNA, 20 bp, Artificial Sequence
  Synthesized
ttgagaatacgcccacgcga

SEQ ID NO: 218 - DNA, 20 bp, Artificial Sequence
  Synthesized
ttgagaatacgcccacgcga

SEQ ID NO: 219 - DNA, 20 bp, Artificial Sequence
  Synthesized
ttgagaatacgcccacgcga

SEQ ID NO: 220 - DNA, 20 bp, Artificial Sequence
  Synthesized
ctgcgatgtcggtttccgcg

SEQ ID NO: 221 - DNA, 20 bp, Artificial Sequence
  Synthesized
ctgagatgtcggtttccgcg

SEQ ID NO: 222 - DNA, 20 bp, Artificial Sequence
  Synthesized
ctgagatgtcggtttccgcg

SEQ ID NO: 223 - DNA, 20 bp, Artificial Sequence
  Synthesized
ctgagatgtcggtttccgcg

SEQ ID NO: 224 - DNA, 20 bp, Artificial Sequence
  Synthesized
ctgagatgtcggtttccgcg

SEQ ID NO: 225 - DNA, 20 bp, Artificial Sequence
  Synthesized
ctgagatgtcggtttccgcg

SEQ ID NO: 226 - DNA, 20 bp, Artificial Sequence
  Synthesized
ctgagatgtcggtttccgcg

SEQ ID NO: 227 - DNA, 20 bp, Artificial Sequence
  Synthesized
ctgagatgtcggtttccgcg

SEQ ID NO: 228 - DNA, 20 bp, Artificial Sequence
  Synthesized
ctgagatgtcggtttccgcg

SEQ ID NO: 229 - DNA, 20 bp, Artificial Sequence
  Synthesized
ctgagatgtcggtttccgcg

SEQ ID NO: 230 - DNA, 20 bp, Artificial Sequence
  Synthesized
ctgagatgtcggtttccgcg

SEQ ID NO: 231 - DNA, 20 bp, Artificial Sequence
  Synthesized
ctgagatgtcggtttccgcg

SEQ ID NO: 232 - DNA, 20 bp, Artificial Sequence
  Synthesized
ctgagatgtcggtttccgcg

SEQ ID NO: 233 - DNA, 20 bp, Artificial Sequence
  Synthesized
ctgagatgtcggtttccgcg

SEQ ID NO: 234 - DNA, 20 bp, Artificial Sequence
  Synthesized
ctgagatgtcggtttccgcg

SEQ ID NO: 235 - DNA, 16 bp, Artificial Sequence
  Synthesized
cgtgtatcaccacgcc

SEQ ID NO: 236 - DNA, 20 bp, Artificial Sequence
  Synthesized
caatacaaagattttcctac

SEQ ID NO: 237 - DNA, 20 bp, Artificial Sequence
  Synthesized
tgagatggagtcttgctgtg

SEQ ID NO: 238 - DNA, 20 bp, Artificial Sequence
  Synthesized
caggcagatcacaaggtcag

SEQ ID NO: 239 - DNA, 20 bp, Artificial Sequence
  Synthesized
tttctttcacagatgtggat

SEQ ID NO: 240 - DNA, 20 bp, Artificial Sequence
  Synthesized
tttatttcacagatgtggat

SEQ ID NO: 241 - DNA, 20 bp, Artificial Sequence
  Synthesized
aggccaatccgcgccggatg

SEQ ID NO: 242 - DNA, 20 bp, Artificial Sequence
  Synthesized
gatcggcctgaactgccagc

SEQ ID NO: 243 - DNA, 20 bp, Artificial Sequence
  Synthesized
gataggcctgaactgccagc

SEQ ID NO: 244 - DNA, 20 bp, Artificial Sequence
  Synthesized
tcccaaagtactgagattac

SEQ ID NO: 245 - DNA, 20 bp, Artificial Sequence
  Synthesized
gtactttcggaggccgaggc

SEQ ID NO: 246 - DNA, 20 bp, Artificial Sequence
  Synthesized
gctttagcgtcttgagtagc

SEQ ID NO: 247 - DNA, 20 bp, Artificial Sequence
  Synthesized
cgccaaagcaggagaatcgc

SEQ ID NO: 248 - DNA, 20 bp, Artificial Sequence
  Synthesized
gctttcgcgtcttgagtagc

SEQ ID NO: 249 - DNA, 16 bp, Artificial Sequence
  Synthesized
cgtgtatcaccacgcc

SEQ ID NO: 250 - DNA, 20 bp, Artificial Sequence
  Synthesized
caatacaaagattttcctac

SEQ ID NO: 251 - DNA, 20 bp, Artificial Sequence
  Synthesized
tgagatggagtcttgctgtg

SEQ ID NO: 252 - DNA, 20 bp, Artificial Sequence
  Synthesized
caggcagatcacaaggtcag

SEQ ID NO: 253 - DNA, 20 bp, Artificial Sequence
  Synthesized
caggtaatgactaagatgac

SEQ ID NO: 254 - DNA, 35 bp, Artificial Sequence
  Synthesized
tcaggctttttttcacagatgtggattggcgataa

SEQ ID NO: 255 - DNA, 35 bp, Artificial Sequence
  Synthesized
tcaggctttctttcacagatgtggattggcgataa

SEQ ID NO: 256 - DNA, 35 bp, Artificial Sequence
  Synthesized
tcaggctttatttcacagatgtggattggcgataa

SEQ ID NO: 257 - DNA, 35 bp, Artificial Sequence
  Synthesized
tcaggctttgtttcacagatgtggattggcgataa

SEQ ID NO: 258 - DNA, 37 bp, Artificial Sequence
  Synthesized
ccgcatccggcgcggattggcctgaactgccagctgg

SEQ ID NO: 259 - DNA, 37 bp, Artificial Sequence
  Synthesized
ccgcatccggcgcggatcggcctgaactgccagctgg

SEQ ID NO: 260 - DNA, 37 bp, Artificial Sequence
  Synthesized
ccgcatccggcgcggataggcctgaactgccagctgg

SEQ ID NO: 261 - DNA, 37 bp, Artificial Sequence
  Synthesized
ccgcatccggcgcggatgggcctgaactgccagctgg

SEQ ID NO: 262 - DNA, 35 bp, Artificial Sequence
  Synthesized
cccgcctcggcctcctaaagtactgagattacagg

SEQ ID NO: 263 - DNA, 35 bp, Artificial Sequence
  Synthesized
cccgcctcggcctcccaaagtactgagattacagg

SEQ ID NO: 264 - DNA, 35 bp, Artificial Sequence
  Synthesized
cccgcctcggcctccgaaagtactgagattacagg

SEQ ID NO: 265 - DNA, 35 bp, Artificial Sequence
  Synthesized
cccgcctcggcctccaaaagtactgagattacagg

SEQ ID NO: 266 - DNA, 37 bp, Artificial Sequence
  Synthesized
ccagctactcaagacgctaaagcaggagaatcgctgg

SEQ ID NO: 267 - DNA, 37 bp, Artificial Sequence
  Synthesized
ccagctactcaagacgccaaagcaggagaatcgctgg

SEQ ID NO: 268 - DNA, 37 bp, Artificial Sequence
  Synthesized
ccagctactcaagacgcgaaagcaggagaatcgctgg

SEQ ID NO: 269 - DNA, 37 bp, Artificial Sequence
  Synthesized
ccagctactcaagacgcaaaagcaggagaatcgctgg